(19) United States
(12) Patent Application Publication
(10) Pub. No.: US 2024/0009457 A1
(43) Pub. Date: Jan. 11, 2024

(54) SYSTEMS AND METHODS FOR FACILITATING MULTISITE PAIRED CORTICOSPINAL-MOTONEURONAL STIMULATION THERAPY

(71) Applicant: Rehabilitation Institute of Chicago d/b/a Shirley Ryan Abilitylab, Chicago, IL (US)

(72) Inventor: Monica Perez, Chicago, IL (US)

(21) Appl. No.: 18/255,866

(22) PCT Filed: Dec. 3, 2021
(86) PCT No.: PCT/US2021/061895

§ 371 Date: Jun. 3, 2023

Related U.S. Application Data
(60) Provisional application No. 63/121,211, filed on Dec. 3, 2020.

(51) Int. Cl. A61N 1/36; A61N 2/00

(52) U.S. Cl. CPC A61N 1/36062; A61N 2/006; A61N 2/002

(57) ABSTRACT

Various embodiments of a system and associated methods for facilitating a noninvasive stimulation protocol that targets corticospinal-motoneuronal synapses of multiple upper and lower limb muscles simultaneously using principles of spike-timing dependent plasticity. The system applies pre-synaptic and post-synaptic stimuli to cortico-neuronal pairs during each session of the stimulation protocol for rehabilitation of multiple peripheral nerves at a time. The system includes a controller that modulates a post-synaptic pulse initiation time such that an interstimulus interval between an arrival time of a pre-synaptic stimulus and an arrival time of a post-synaptic stimulus is within a predetermined range.

### CROSS-REFERENCE TO RELATED APPLICATIONS

This is a PCT application that claims benefit to U.S. Provisional Patent Application Ser. No. 63/121,211 filed Dec. 3, 2020, which is herein incorporated by reference in its entirety.

### FIELD

The present disclosure generally relates to spinal cord rehabilitation control systems, and in particular, to a system and associated method for facilitating multisite paired corticospinal-motoneuronal stimulation (MPCMS) therapy.

### BACKGROUND

Spinal cord injuries (SCI) can involve weakening of an electrical connection between a pre-synaptic cell (corticospinal neuron) and a post-synaptic cell (peripheral motor neuron) of a corticospinal motoneuronal pairing. The pre-synaptic cell is a corticospinal neuron that has its body in the cortex and its axon through the spinal cord where it makes connections (synapses) to the post-synaptic cell, the peripheral motor neuron. Synaptic connections can, in some instances, be strengthened in vitro through repeated electrical stimulation to the pre-synaptic cell and the post-synaptic cell. However, translating this in vivo remains a challenge. There is a need for a system that can effectively boost residual corticospinal connections in paralyzed or partially paralyzed subjects at multiple locations to augment exercise-mediated recovery in humans with different levels of SCI.

It is with these observations in mind, among others, that various aspects of the present disclosure were conceived and developed.

### BRIEF DESCRIPTION OF THE DRAWINGS

FIG. 1 is a simplified diagram showing a system for facilitating MPCMS therapy;

FIGS. 2A and 2B are simplified diagrams showing connection of the body with the system of FIG. 1;

FIG. 3 is a simplified illustration showing MPCMS stimulation of a synapse by the system of FIG. 1;

FIG. 4 is a process flow showing a method for facilitating MPCMS therapy by the system of FIG. 1;

FIG. 5 is a simplified diagram showing an example computing system for implementation of the system of FIG. 1;

FIG. 6 is an illustration showing placement of pre- and post-synaptic stimuli for facilitation of MPCMS protocol using the system of FIG. 1 according to a second validation study;

FIGS. 7A-7F is a series of photographs showing exercise according to the MPCMS protocol; and

FIG. 8 is a diagram illustrating facilitation of MPCMS protocol for the second validation study;

FIGS. 9A-D are a series of graphical representations showing MEP, C-root, M-wave and other results for biceps brachii;

FIGS. 10A-D are a series of graphical representations showing MEP, C-root, M-wave and other results for first dorsal interosseous;

FIGS. 11A-D are a series of graphical representations showing MEP, C-root, M-wave and other results for quadriceps;

FIGS. 12A-D are a series of graphical representations showing MEP, C-root, M-wave and other results for tibialis anterior;

FIGS. 13A-D are a series of graphical representations showing raw MEP traces for various muscle groups before, following 20 sessions, and following 40 sessions;

FIGS. 14A-D are a series of graphical representations showing rectified electromyographic traces during MVCs for various muscle groups before, following 20 sessions, and following 40 sessions;

FIGS. 15A and 15B are graphical representations showing sensory outcomes prior to and following 40 sessions;

FIGS. 16A-16C are graphical representations showing motor outcome scores prior to and following 40 sessions;

FIGS. 17A and 17B are graphical representations and related images showing functional outcome scores including GRASSP and 10-m walk prior to and following 40 sessions; and

FIG. 18 is a graphical representation showing quality of life improvement scores following 40 sessions.

Corresponding reference characters indicate corresponding elements among the view of the drawings. The headings used in the figures do not limit the scope of the claims.

### DETAILED DESCRIPTION

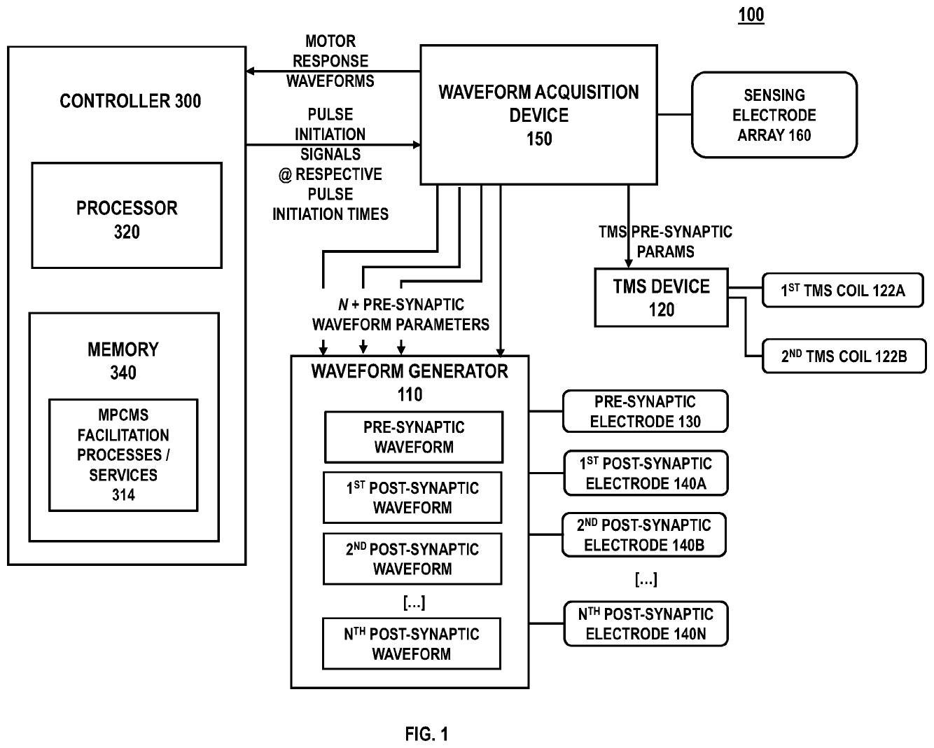

Various embodiments of a system that engages residual neuronal networks in humans with spinal cord injuries by facilitating multisite paired corticospinal-motoneuronal stimulation (MPCMS) therapy are described herein. In MPCMS, corticospinal electrical volleys (pre-synaptic stimulus) evoked by transcranial magnetic stimulation (TMS) over the primary motor cortex or electrical stimulation (ES) over the spine are timed to arrive at corticospinal-motoneuronal synapses of limb muscles before or after antidromic potentials (post-synaptic stimulus) elicited in motoneurons by electrical stimulation of a peripheral nerve. MPCMS likely elicits spike-timing dependent plasticity (STDP) changes at spinal synapses of somatic motoneurons. The system described herein applies a pre-synaptic stimulus to a pre-synaptic cell of a corticospinal-motoneuronal pairing, and subsequently applies a post-synaptic stimulus to a post-synaptic cell of the corticospinal-motoneuronal pairing such that the pre-synaptic stimulus from the cortex arrives at a synapse of the corticospinal-motoneuronal pairing a predetermined time interval, preferably 1-2 ms, before the post-synaptic stimulus from the peripheral nerve. In some embodiments, the system can apply stimulus to target multiple muscle groups at a time through more than one peripheral nerve to improve patient outcomes. Referring to the drawings, embodiments of a system for facilitating MPCMS therapy are illustrated and generally indicated as 100 in FIGS. 1-18.

Referring to FIGS. 1-5, an embodiment of the system 100 includes a controller 300 in electrical communication with a transcranial magnetic stimulation (TMS) device 120 for generating a pre-synaptic stimulus for application to a body during multisite paired corticospinal-motoneuronal stimulation (MPCMS) therapy. Further, the system 100 includes a waveform generator 110 for generating a plurality of post-synaptic stimuli for application to the body during MPCMS therapy. In some embodiments, the waveform generator 110 can also generate an additional pre-synaptic stimulus for application to the body during MPCMS therapy, as will be described in greater detail herein. As specifically illustrated in FIGS. 1-3, the system 100 includes one or more TMS coils 122 of a TMS device 120 configured to induce or otherwise apply the pre-synaptic stimulus to a motor cortex of a body according to TMS pre-synaptic parameters including pulse initiation time provided to the TMS device 120 by the controller 300. Similarly, the system 100 includes a plurality of post-synaptic electrodes 140 in communication with the waveform generator 110 configured to induce or otherwise apply the post-synaptic stimuli to a plurality of peripheral nerves of the body according to a plurality of post-synaptic waveform parameters including pulse initiation time provided to the waveform generator 110 by the controller 300. In some embodiments, the system 100 is configured to apply an additional pre-synaptic stimulus to the thoracic spine through a pre-synaptic electrode 130 in communication with the waveform generator 110 based on pre-synaptic parameters including pulse initiation time provided by the controller 300. In practice, the additional pre-synaptic stimulus is applied to the thoracic spine to aid in application of MPCMS therapy to the lower body.

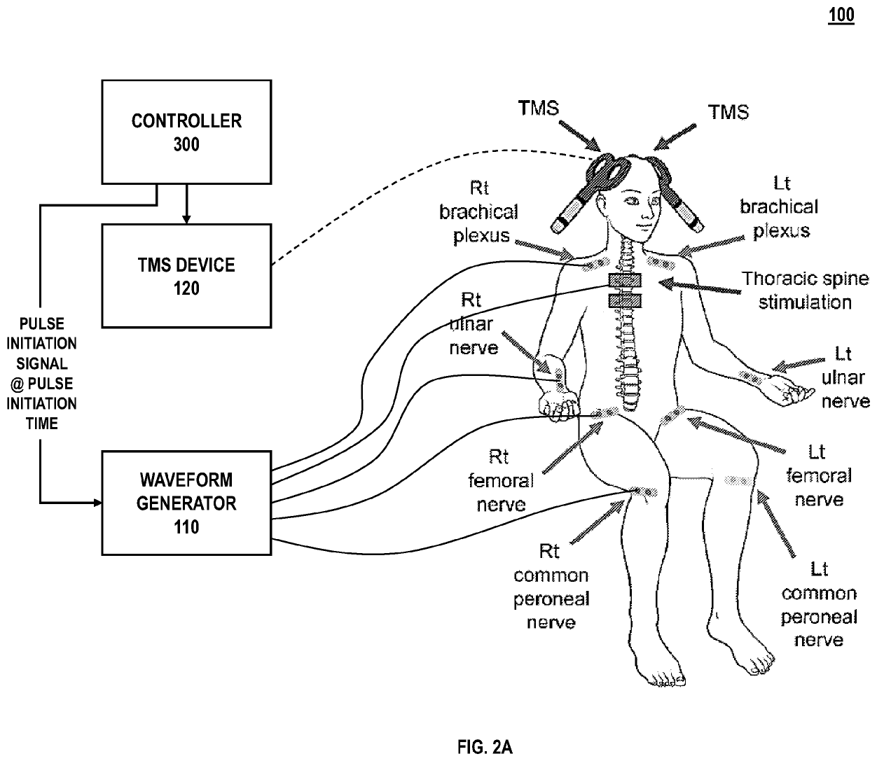

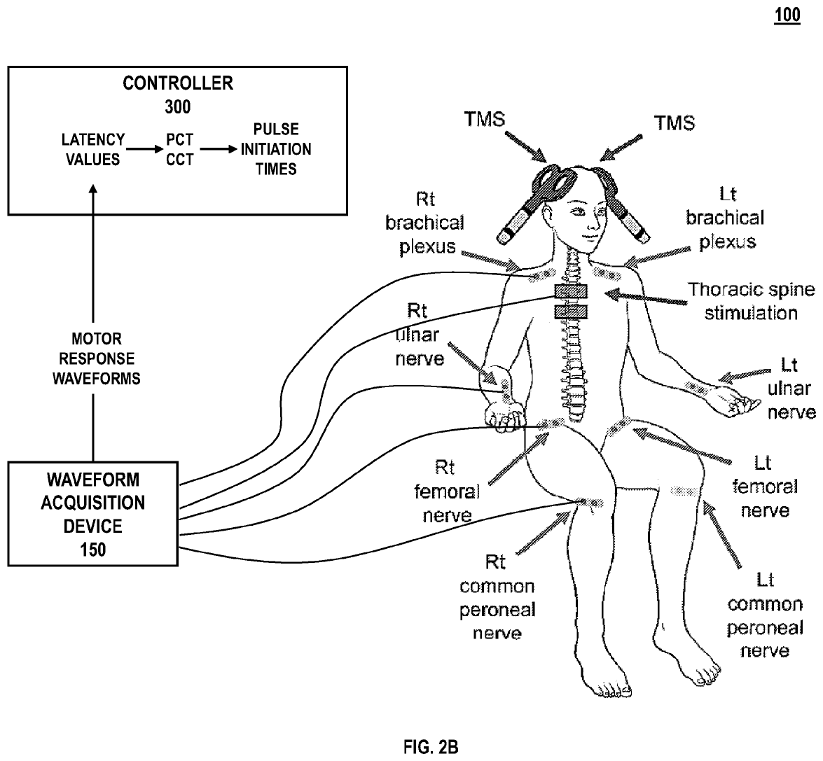

Referring directly to FIG. 2A, TMS is applied cranially and induces action potentials (pre-synaptic stimulus) within the corticospinal pathway. Combined pre-synaptic stimuli and post-synaptic stimuli can aid in therapeutic restoration of function in targeted muscles. Thus, the controller 300 modulates or otherwise maintains control of pre-synaptic waveforms and post-synaptic waveforms representative of pre-synaptic stimuli and post-synaptic stimuli to be generated by the waveform generator 110. The waveform generator 110 individually applies electrical current to each peripheral nerve of the plurality of peripheral nerves (or to the thoracic spine) according to associated waveform parameters for application of the post-synaptic stimuli (peripheral nerve) or pre-synaptic stimulus (thoracic spine) to a plurality of corticospinal-motoneuronal pairs. Similarly, the TMS device 120 induces an action potential as a pre-synaptic stimulus within the brain for application of the pre-synaptic stimulus to each corticospinal-motoneuronal pair. Empirical evidence has demonstrated that an optimal interstimulus interval between a pre-synaptic time-of-arrival of the pre-synaptic stimulus and a respective post-synaptic time-of-arrival of each post-synaptic stimulus is between 1-2 milliseconds for each grouping of stimuli applied. Thus, the controller 300 adjusts pre-synaptic and post-synaptic pulse initiation times provided to the waveform generator 110 to accommodate for differences in nerve length between various peripheral nerves such that the post-synaptic stimuli arrives at a synapse of the corticospinal-motoneuronal pair 1-2 milliseconds after the pre-synaptic stimuli.

### Pre-Synaptic Stimuli

As discussed, the system 100 applies or otherwise induces a pre-synaptic stimulus to the motor cortex which terminates at a pre-synaptic cell of a corticospinal-motoneuronal pair. The pre-synaptic stimulus is applied or induced within the motor cortex which causes an action potential to propagate orthodromically down an axon of the pre-synaptic cell. The pre-synaptic stimulus induces an action potential in the pre-synaptic axon (corticospinal neuron) and is paired with a post-synaptic stimulus to an associated peripheral nerve (spinal-motoneuron). Pre-synaptic stimuli can be applied or induced in at least two ways:

1. TMS.

TMS can be used to induce the pre-synaptic stimulus by applying a magnetic field parallel to the skull. This stimulates an electrical field perpendicular to the skull, which in turn triggers an action potential (electrical impulse) in a corticospinal neuron that propagates down through the spinal cord and connects to a peripheral motor nerve in the spinal cord. Referring to FIG. 1, the controller 300 provides TMS pre-synaptic parameters including pulse initiation time to the TMS device 120 to generate a magnetic field within one or more TMS coils 122 of the TMS device 120. In the examples shown, the system 100 includes a first TMS coil 122A and a second TMS coil 122B of the one or more TMS coils 122 that apply the magnetic field to a respective left side and right side of the skull. This induces the action potential (pre-synaptic stimulus) that propagates down the corticospinal neuron and to the synapse.

2. Direct Thoracic Stimulation.

Further, in some embodiments, the waveform generator 110 can additionally apply a pre-synaptic stimulus to the thoracic spine. The controller 300 provides pre-synaptic waveform parameters including pulse initiation time to the waveform generator 110 to apply a current corresponding with the pre-synaptic waveform parameters to the corticospinal neuron through a pre-synaptic electrode 130 in communication with the waveform generator 110. This induces the action potential (pre-synaptic stimulus) that propagates down the corticospinal neuron and to the synapse.

### Post-Synaptic Stimuli

As further shown in FIG. 2A, the system 100 is configured to apply post-synaptic stimuli to a plurality of peripheral nerves at a time. In the example shown, post-synaptic stimuli are applied from the waveform generator 110 to eight separate locations on the body; particularly to peripheral limbs such as right and left common peroneal nerves, right and left femoral nerves, right and left ulnar nerves, and right and left brachial plexus nerves that communicate with the spinal cord. Each location requires different waveform parameters including pulse initiation time to arrive at the synapse at the proper time due to physiological length of the associated peripheral nerve. The system 100 applies post-synaptic stimuli to the peripheral limbs through N post-synaptic electrodes 140A-140N in communication with the waveform generator 110, where N is the number of peripheral nerves to be stimulated. The waveform generator 110 generates N post-synaptic stimuli at N post-synaptic electrodes 140A-140N. The controller 300 provides N post-synaptic waveform parameters to the waveform generator 110 to apply current corresponding with the N post-synaptic waveform parameters to associated peripheral nerves through respective post-synaptic electrodes 140A-140N in communication with the waveform generator 110. This induces the action potential (post-synaptic stimulus) that propagates up the peripheral nerve and to the synapse. Each set of post-synaptic waveform parameters includes a respective post-synaptic pulse initiation time that is specific to the associated peripheral nerve to ensure that the post-synaptic stimulus arrives at the synapse 1-2 ms after the associated pre-synaptic stimulus arrives.

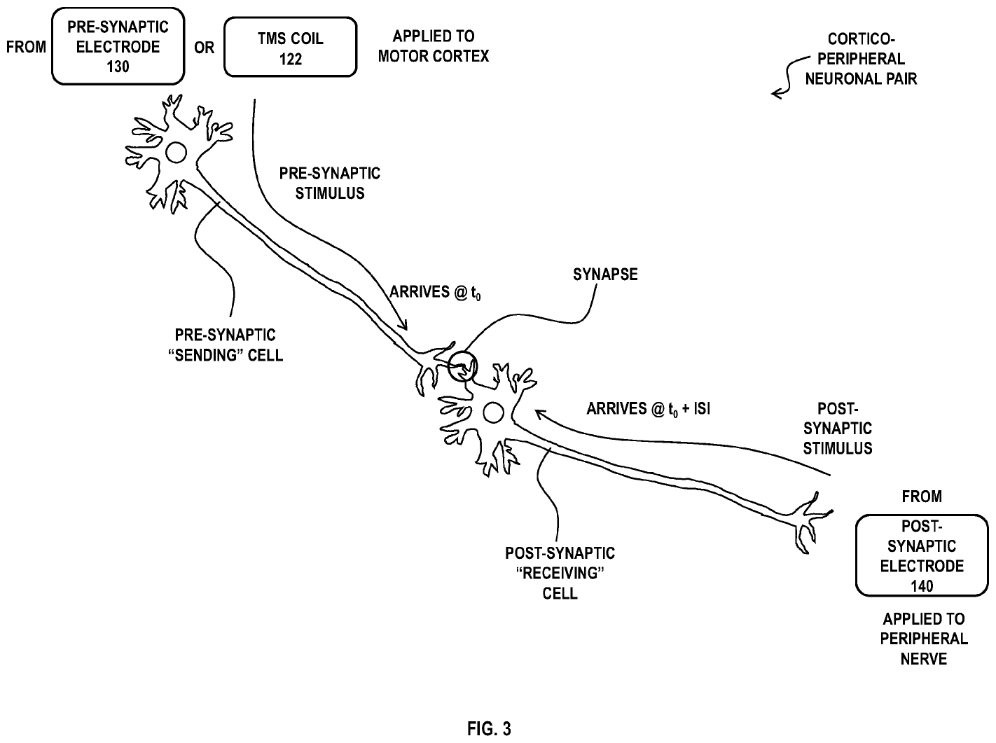

FIG. 3 illustrates a corticospinal-motoneuronal neuronal pair including a pre-synaptic cell in association with the motor cortex (corticospinal neuron) and a post-synaptic cell in association with the peripheral nerve. A junction of the two is illustrated at the synapse. The system 100 facilitates MPCMS therapy to restore corticospinal-motoneuronal nerve function by first stimulating the corticospinal neuron by applying the pre-synaptic stimulus to the pre-synaptic cell through a pre-synaptic electrode 130 or through a TMS coil 122. The system 100 subsequently stimulates the peripheral nerve by applying post-synaptic stimulus in the form of simple electrical pulses to major peripheral nerves in the limbs through one or more post-synaptic electrodes 140. For effective MPCMS, pre-synaptic pulse initiation time and post-synaptic pulse initiation time are important. They must occur such that the signal from the cortex arrives at the synapse 1-2 ms before the signal from the peripheral nerve. So, the electrical pulse is applied to the peripheral nerves following a delay after the stimulation to the cortex. The length of the delay is dependent upon a length of the peripheral nerve. (i.e. if the pre-synaptic stimulus arrives at time to, then the post-synaptic stimulus must arrive at time t₀+[1 ms, 2 ms]).

The controller 300 manages application of pre-synaptic and post-synaptic stimuli to the body by providing control inputs to the TMS device 120 and waveform generator 110. In particular, the controller 300 determines and communicates waveform parameters including the first pre-synaptic and post-synaptic pulse initiation times that the interstimulus interval is preferably 1-2 ms. In other embodiments, the interval may differ and can be greater than Oms and less than 5 ms. During MPCMS, the system 100 delivers 180 pairs of pre-synaptic and post-synaptic stimuli every 10 seconds (˜30 min, 0.1 Hz), where corticospinal volleys (pre-synaptic stimuli) evoked by TMS over the primary motor cortex are timed to arrive at corticospinal-motoneuronal synapses of each muscle ˜1-2 ms before the post-synaptic antidromic potentials evoked in motoneurons by peripheral nerve stimulation (PNS).

Referring to FIGS. 1-4, for MPCMS facilitation, in a first step, two or more peripheral nerves innervating at least two different targeted muscle sites in the subject and forming two or more peripheral nerve-muscle pairings must be identified. This involves identifying two or more corticospinal-motoneuronal connections, each comprising a corticospinal neuron connected at a synapse with each peripheral nerve in each of the peripheral nerve-muscle pairings. Once the appropriate peripheral nerve-muscle pairings have been identified, the system 100 acquires a plurality of latency values associated with the targeted peripheral nerves and the motor pathway. In some embodiments, this can be achieved using a waveform acquisition device 150 such as a Power 1401 acquisition interface that is operable for obtaining a plurality of motor response waveforms including MEP, F-wave, and M-max waveforms for the body. In some embodiments, the waveform acquisition device 150 acquires the plurality of motor response waveforms from the body through a sensing electrode array 160 that includes a plurality of electrodes in communication with the two or more peripheral nerves and the motor pathway. The controller 300 determines or otherwise obtains the associated plurality of latency values including MEP, F-wave, and M-max latencies. The controller 300 then uses the plurality of latency values to calculate a peripheral conduction time (PCT) and a central conduction time (CCT) for each of the peripheral nerve-muscle pairings. PCT is the amount of travel time necessary for a post-synaptic stimulus to arrive at the synapse when applied to a location along the peripheral nerve. Likewise, CCT is the amount of travel time necessary for a pre-synaptic stimulus to arrive at the synapse when applied to a location along the cortical or motor pathway nerve. The controller 300 then adjusts waveform parameters including a pulse initiation time for each pre-synaptic stimulus and post-synaptic stimulus based on the calculated PCT and CCT. The system 100 then applies, based on the waveform parameters, a resultant pre-synaptic stimulus and the post-synaptic stimuli that arrive at the synapse within the appropriate interstimulus interval.

PCT. The values to calculate PCT for a peripheral nerve-muscle pairing are found using a plurality of latency values from the plurality of motor response waveforms including MEPs, F-wave, and M-max that are recorded by the system 100 for the body. MEP latencies are recorded during isometric ˜10% of MVC of the target muscle to determine the shortest and clearest response for estimations. The onset latency is defined as the time when each response exceeded 2 SD of the mean rectified pre-stimulus activity (100 ms) in the averaged waveform. Peripheral conduction time (PCT) is calculated using the following equation:

PCT=(F-wave latency−M-max latency)×0.5

CCT. Central conduction time (CTT) was calculated using the following equation:

CCT=MEP latency−(PCT+M-max latency)

Alternatively, the latency of H-reflex can be used instead when it is difficult to elicit F-waves. When it was not possible to record F-waves or H-reflex (i.e. biceps brachii), then C-roots can be stimulated with TMS at cervical spinous processes C5-6. Then, CCT is calculated by adding to the latency from TMS of the C-root to 1.5 ms [estimated time of synaptic transmission plus conduction to the nerve root at the vertebral foramina] and subtracting from the MEP latency [MEP−(C-root+1.5)]. PCT is calculated by subtracting the M-max latency from the C-root latency and adding 0.5 ms, the estimated time of antidromic conduction time from the vertebral foramina to the dendrites [(C-root−M-max)+0.5)].

Adjusting Pulse Initiation Time. Following determination of PCT and CCT, the controller 300 determines a pulse initiation time for each pre-synaptic and post-synaptic stimulus to be applied such that the pulse arrival time for the associated stimulus is within the appropriate interstimulus interval relative to one another.

For instance, given a goal interstimulus interval (ISI), a calculated CCT and a calculated PCT for a particular peripheral nerve of the plurality of peripheral nerves, the controller 300 determines a delay interval at which a pre-synaptic pulse initiation time of the pre-synaptic stimulus is delayed relative to a post-synaptic pulse initiation time of the post-synaptic stimulus to account for differences in conduction time between different nerves.

delay interval=PCT−CCT−ISI

pre-synaptic pulse initiation time=ISI+CCT−delay interval=delay interval+post-synaptic pulse initiation time

For example, consider a calculated PCT value of 7 ms delay before arrival of the post-synaptic stimulus at the synapse and a calculated CCT value of 3 ms delay before arrival of the post-synaptic stimulus at the synapse. To arrive within an interstimulus interval of 1.5 ms, the post-synaptic stimulus would need to be initiated 2.5 ms before initiation of the pre-synaptic stimulus (delay interval=2.5 ms). In most scenarios, the PCT value is longer than that of the CCT value and the interstimulus interval combined. In such a situation, the controller 300 initiates the post-synaptic stimulus at the post-synaptic initiation time and then initiates the pre-synaptic stimulus afterward at the pre-synaptic initiation time, which would be at the post-synaptic pulse initiation time followed by the delay interval. In other scenarios in which the CCT value combined with the interstimulus interval are smaller than the PCT value, then an initiation order between the post-synaptic stimulus and the pre-synaptic stimulus would be reversed. The controller 300 initiates the pre-synaptic stimulus at the pre-synaptic initiation time and then initiates the post-synaptic stimulus afterward at the post-synaptic initiation time, which would be at the pre-synaptic pulse initiation time followed by the delay interval.

Extending this logic to the multiple peripheral limbs to be stimulated, the controller 300 selects an optimal post-synaptic pulse initiation time of each individual post-synaptic stimulus such that the interstimulus interval between the pre-synaptic time of arrival and each respective post-synaptic time of arrival at a synapse is within 1-2 ms. Further, the post-synaptic pulse initiation times can be set and the controller 300 can select an optimal pre-synaptic pulse initiation time such that the interstimulus interval between the pre-synaptic time of arrival and each respective post-synaptic time of arrival at a synapse is preferably within 1-2 ms.

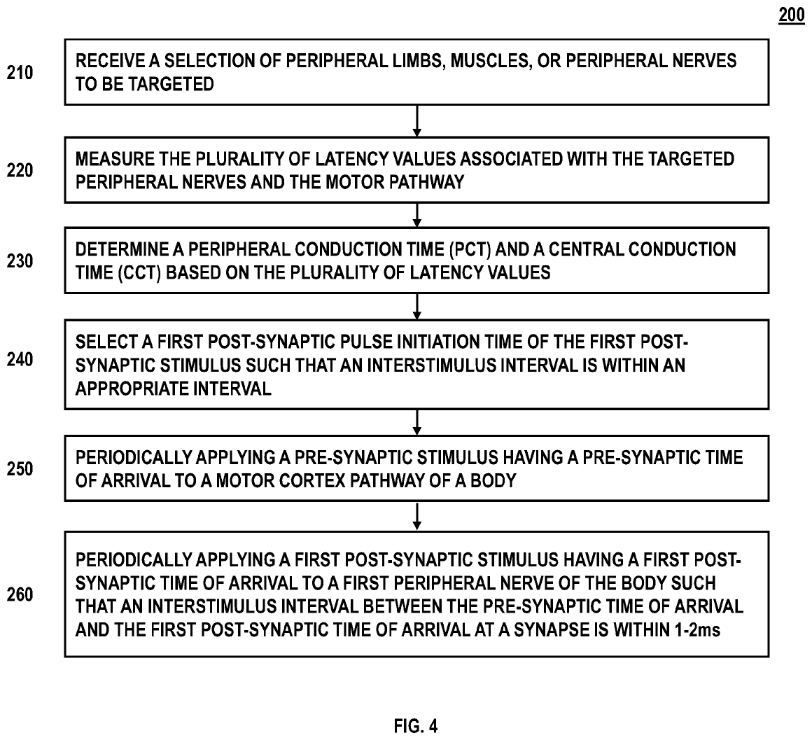

Referring to FIG. 4, a process flow 200 is illustrated for execution by the controller 300 of the system 100. At block 210, the controller 300 receives a selection of peripheral limbs, muscles, or peripheral nerves to be targeted. At block 220, the controller measures the plurality of latency values associated with the targeted peripheral nerves and the motor pathway through recordation of a plurality of motor response waveforms from which the plurality of latency values are extracted. As discussed above, this can be achieved using the waveform acquisition device 150 that is operable for obtaining a plurality of motor response waveforms including MEP, F-wave, and M-max waveforms for the body. The controller 300 determines or otherwise obtains the associated plurality of latency values including MEP, F-wave, and M-max latencies from the waveform acquisition device 150. At block 230, the controller 300 determines a peripheral conduction time (PCT) and a central conduction time (CCT) based on the plurality of latency values. At block 240, the controller 300 selects a post-synaptic pulse initiation time of the post-synaptic stimulus such that the interstimulus interval between the pre-synaptic time of arrival and the post-synaptic time of arrival at a synapse is within the appropriate interval.

At block 250, the controller 300 periodically applies the pre-synaptic stimulus having the pre-synaptic time of arrival from the motor cortex to the spinal cord. This is achieved as described above using TMS device 120 or using waveform generator 110 to apply or otherwise induce the pre-synaptic stimulus to the motor cortex (corticospinal neuron). In some embodiments, the waveform acquisition device 150 can additionally aid in facilitating communication with the waveform generator 110 and the TMS device 120. The controller 300 can provide pulse initiation signals at respective pulse initiation times to the waveform acquisition device 150 that instructs the waveform generator 110 to generate associated waveforms according to the waveform parameters at the pulse initiation time dictated by the pulse initiation signal. At block 260, the controller 300 periodically applies the post-synaptic stimulus having the post-synaptic time of arrival to the peripheral nerve of the body. This is achieved as described above using waveform generator 110 to apply the post-synaptic stimulus to the peripheral nerve.

In one embodiment of the system 100, a Power 1401 acquisition interface from Cambridge Electric Design is used in communication with the controller 300 as the waveform acquisition device 150 to obtain the plurality of latency values and also to act as the waveform generator 110 to trigger several electrical stimulators (in one example, a plurality of Digitimer DS7R stimulators) and TMS devices using a customized cable and a written configuration that contains 11 states as follow:

State 1: STDP (all sites are triggered at specific times according to their individual PCT or CCT values)

State 2: A pulse initiation signal to stimulate the right brachial plexus

State 3: A pulse initiation signal to stimulate the right ulnar nerve State 4: A pulse initiation signal to stimulate the right femoral

nerve

State 5: A pulse initiation signal to stimulate the right common peroneal nerve

State 6: A pulse initiation signal to stimulate the left brachial

plexus

State 7: A pulse initiation signal to stimulate the left ulnar nerve State 8: A pulse initiation signal to stimulate the left femoral

nerve

State 9: A pulse initiation signal to stimulate the left common peroneal nerve

State 10: Thoracic electrical stimulation

State 11: TMS

Each state has a duration of 10 seconds and a predefined pulse initiation time at which the stimulation is triggered by communication of the pulse initiation signal from the controller 300. All pulse initiation times for each pulse initiation signal within each state are adjusted depending on specific CCT and PCT values defined during the assessments. It should be noted that multiple states can be triggered at a time, and that alternative peripheral nerves or limbs can be selected as well.

Depending on the number of targeted peripheral nerves, the controller 300 periodically applies additional post-synaptic stimuli having post-synaptic times of arrival to additional peripheral nerves within the body. In a therapeutic setting, it is highly recommended that the subject exercise affected peripheral limbs immediately following MPCMS application to improve results.

### Computer-Implemented System

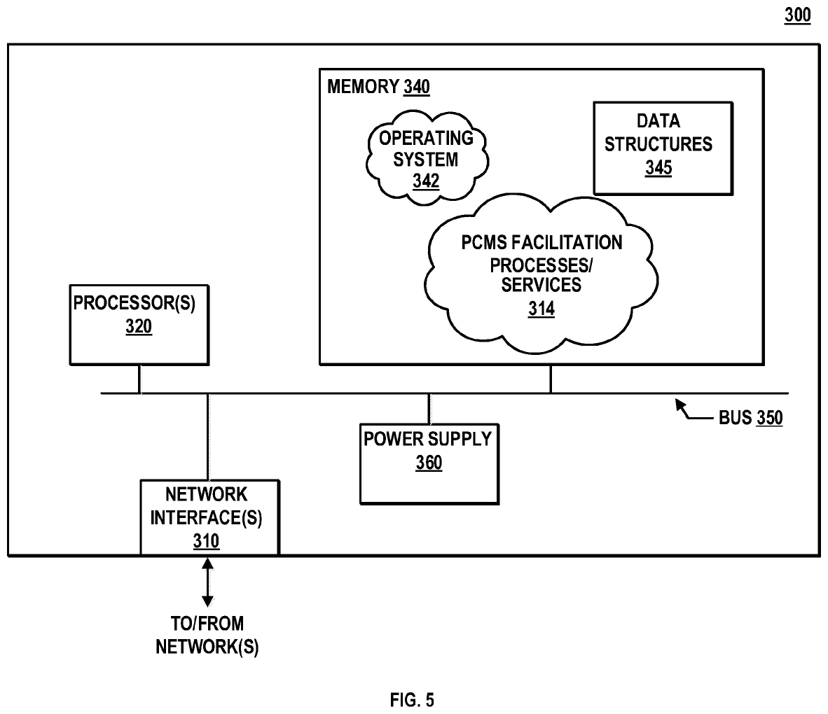

FIG. 5 is a schematic block diagram of an example device 300 that may be used with one or more embodiments described herein, e.g., as a component of system 100 shown in FIG. 1.

Device 300 comprises one or more network interfaces 310 (e.g., wired, wireless, PLC, etc.), at least one processor 320, and a memory 340 interconnected by a system bus 350, as well as a power supply 360 (e.g., battery, plug-in, etc.).

Network interface(s) 310 include the mechanical, electrical, and signaling circuitry for communicating data over the communication links coupled to a communication network. Network interfaces 310 are configured to transmit and/or receive data using a variety of different communication protocols. As illustrated, the box representing network interfaces 310 is shown for simplicity, and it is appreciated that such interfaces may represent different types of network connections such as wireless and wired (physical) connections. Network interfaces 310 are shown separately from power supply 360, however it is appreciated that the interfaces that support PLC protocols may communicate through power supply 360 and/or may be an integral component coupled to power supply 360.

Memory 340 includes a plurality of storage locations that are addressable by processor 320 and network interfaces 310 for storing software programs and data structures associated with the embodiments described herein. In some embodiments, device 300 may have limited memory or no memory (e.g., no memory for storage other than for programs/processes operating on the device and associated caches).

Processor 320 comprises hardware elements or logic adapted to execute the software programs (e.g., instructions) and manipulate data structures 345. An operating system 342, portions of which are typically resident in memory 340 and executed by the processor, functionally organizes device 300 by, inter alia, invoking operations in support of software processes and/or services executing on the device. These software processes and/or services may include MPCMS facilitation processes/services 314 described herein. Note that while MPCMS facilitation processes/services 314 is illustrated in centralized memory 340, alternative embodiments provide for the process to be operated within the network interfaces 310, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the MPCMS facilitation processes/services 314 is shown as a standalone process, those skilled in the art will appreciate that this process may be executed as a routine or module within other processes.

### Method of Treatment

In accordance with some aspects of the present disclosure, a method of treating a subject is also provided. The method comprises (a) identifying two or more peripheral nerves innervating at least two different muscle sites in the subject and forming two or more peripheral nerve-muscle pairings; (b) identifying two or more corticospinal-motoneuronal connections each comprising a corticospinal neuron connected at a synapse with each peripheral nerve in each of the peripheral nerve-muscle pairings; (c) calculating a peripheral conduction time (PCT) and a central conduction time (CCT) for the each of the peripheral nerve-muscle pairings; (d) periodically applying a first stimulus to a location in the central nervous system (CNS) in the subject such that the first stimulus triggers a descending signal in at least one corticospinal neuron in the corticospinal-motoneuron connections; and (e) periodically applying a second stimulus to each of the two or more peripheral nerves such that the second stimulus triggers an ascending signal in the each of the two or more peripheral nerves, wherein, each ascending signal and each descending signal arrive at the synapse of each corticospinal-motoneuronal connections and the descending signal arrives at a pre-determined interstimulus interval (ISI) prior to the arrival of the ascending signal.

In various methods, the two or more peripheral nerve-muscle pairings may comprise one or more peripheral nerves selected from the group consisting of brachial plexus, ulnar nerve, femoral nerve, and common peroneal nerve. In various methods, the two or more peripheral nerve-muscle pairings may comprise two or more peripheral nerves selected from the group consisting of brachial plexus, ulnar nerve, femoral nerve, and common peroneal nerve.

In various embodiments, the peripheral conduction time (PCT) for each peripheral nerve-muscle pairing is calculated using the following equation: PCT=(F-wave latency−M-max latency)×0.5. In some embodiments, the central conduction time (CCT) for each peripheral nerve-muscle pairing is calculated using the following equation: CCT=MEP latency−(PCT+M-max latency).

In various aspects, the first stimulus may be applied using transcranial magnetic stimulation. In other aspects, the first stimulus may be applied using thoracic spinal stimulation. In any of these embodiments, the second stimulus may be applied using electrical stimulation.

In various aspects, the interstimulus interval (ISI) is about 0-5 milliseconds. For example, the interstimulus interval (ISI) may be about 1 to 2 milliseconds. In some aspects, paired sets of first and second stimuli are applied at a frequency of about 0.1 Hz for about 30 seconds.

In any of the methods of treatment provided herein, the subject is paralyzed, partially paralyzed and/or have or have had a spinal cord injury (e.g., a cervical spinal cord injury). In some aspects, the subject is a mammal (e.g., a human).

In accord with various aspects of the present disclosure, the methods of treatment provided herein may be performed using any of the systems or controllers described herein.

### Validation Study

The below validation study (Noninvasive-Multisite Corticospinal Synaptic Plasticity Restores Arm and Leg Function in Humans with Chronic Tetraplegia) is included herein to provide additional practical implementation details and clinical results for application of MPCMS therapy using embodiments of the system 100.

The validation study includes an embodiment of the system 100 that first stimulates the cortex in more than one area, and then stimulates more than one peripheral nerve. The system 100 applies or otherwise induces the pre-synaptic stimulus to the motor cortex, specifically the portion of motor cortex that controls the peripheral limb of interest, through the TMS device 110 that applies a magnetic field parallel to the skull and triggers an action potential (electrical impulse) in a corticospinal neuron that stretches down through the spinal cord and connects to a peripheral motor nerve in the spinal cord. In some embodiments, two TMS coils 122A and 122B are used to induce the action potentials on either side of the skull. The validation study further includes application of the additional pre-synaptic stimulus in the form of simple electrical pulses to the thoracic spine using the waveform generator 110 in communication with a pre-synaptic electrode 130 to aid in rehabilitation of the lower body. Further, the system 100 applies the post-synaptic stimulus in the form of simple electrical pulses to a plurality of peripheral nerves in the peripheral limbs of interest through the waveform generator 110. Each peripheral nerve receives its own signal from a respective post-synaptic electrode 140. For N peripheral nerves to be stimulated, N post-synaptic electrodes 140A-N are provided. The system 100 applies the post-synaptic stimuli such that the pre-synaptic stimulus from the cortex arrives at the synapse 1-2 ms before each post-synaptic stimulus from the peripheral nerves. In particular, the electrical pulse is applied to the peripheral nerves after a delay after the stimulation to the cortex. Subjects were then required to exercise and results were collected after several sessions.

Validation Study: Noninvasive-Multisite Corticospinal Synaptic Plasticity Restores Arm and Leg Function in Humans with Chronic Tetraplegia

Cervical spinal cord injury (tetraplegia) causes permanent deficits in the control of voluntary movement of the arms and legs. Voluntary movement depends on the efficacy of synapses between corticospinal axons and spinal motor neurons. This validation study developed a noninvasive stimulation protocol that targets corticospinal-motoneuronal synapses of multiple upper and lower limb muscles simultaneously using principles of spike-timing dependent plasticity facilitated by the system 100 (FIGS. 1-5). After 40 sessions over 8 weeks of targeted multisite stimulation, combined with standard rehabilitation, nine tetraplegic patients with permanent deficits in arm and leg function (1-27 years) exhibited a twofold increase in grasping, overground walking ability, and quality of life outcomes. One of the patients that could not walk at the start of the protocol progressed to walking several steps independently with the support of an assistive device. Electrophysiological responses elicited by stimulation of corticospinal axons increased in size in all targeted upper and lower limb muscles, suggesting a spinal origin for this plasticity. These results demonstrate for the first time that a noninvasive method that strengthen corticospinal synaptic transmission at multiple sites for 40 sessions in 8-weeks can recover arm and leg function simultaneously in humans with long-term tetraplegia.

### Introduction

Cervical spinal cord injury (SCI) or tetraplegia is the most frequent neurological category reported in humans. Tetraplegia disrupts connections from the central nervous system to upper and lower limb muscles leading to simultaneous deficits in daily life functions such as grasping and walking. In humans, the use of exercise combined with either epidural or transcutaneous electrical stimulation of the spinal cord showed substantial restoration in the ability to grasp and walk after SCI. Although there is a paucity of studies on transcutanueous stimulation applied at cervical spinal cord to target upper limb function, epidural stimulation approaches in humans have been predominantly applied at lumbar spinal cord to target the lower limb function. Epidural lumbar spinal cord stimulation showed that some of the most substantial restoration in the ability to walk after SCI. The efficacy has been demonstrated in motor complete SCI, leading to the recovery of independent stepping in the presence of epidural stimulation. Similar results have been attained in incomplete cervical SCI subjects and additionally voluntary control of previously paralyzed lower limb muscles without stimulation has been reported. Although these approaches are beneficial, the epidural stimulation requires a surgical procedure and needed a large number of sessions (>100) to show the reported effects on motor function. There is a need to develop interventions that can more effectively engage spared neural connections in both upper and lower limbs to further improve voluntary motor function in tetraplegia.

Voluntary motor function is largely controlled by the corticospinal tract, which is a major descending motor pathway in mammals. A role of the corticospinal tract in functional recovery after SCI has been proposed for animals and humans. Corticospinal transmission largely depends on the strength of synaptic connections between corticospinal drive and spinal motoneurons. Therefore, strengthening of synaptic connections after SCI would be critical to maximize the transmission of descending command through residual corticospinal tract to the motoneurons. Long-lasting potentiation of synaptic strength can be induced by precisely timing the arrival of presynaptic action potentials prior to postsynaptic depolarizing action potentials (a process known as spike timing-dependent plasticity (STDP)), which was previously showed to enhance voluntary motor output when targeting the spinal cord in intact humans. More recently, a paired stimulation technique based on STDP was combined with exercise in individuals with chronic incomplete SCI showed that corticospinal drive and maximal voluntary contraction (MVC) in the targeted muscle as well as functional outcomes increased after 10 sessions and preserved for up to six months.

Here, the development of a non-invasive stimulation protocol that targeted synapses between corticospinal axons and spinal motoneurons of multiple upper and lower limb muscles simultaneously by using principle of STDP is reported. It was hypothesized that multisite corticospinal-motor neuronal plasticity would enable voluntary locomotion despite chronic paralysis, and that the ability to sustain active movements during training would promote meaningful functional improvements with and even without stimulation. To test this hypothesis, individuals with chronic incomplete SCI underwent 40 sessions of multi-site MPCMS combined with exercise training. It was found that corticospinal drive in all targeted muscles increased to greater extent after multi-site MPCMS. Maximal voluntary contraction in all targeted muscles increased after MPCMS in all targeted muscles, which was also reflected in increase in AIS scores. Behavioral effects were preserved for 6-months as well as self-reported functional changes for walking. This validation study suggests that targeting multiple spinal synapses is an effective strategy to facilitate and preserve motor functional recovery in humans with SCI.

### Results

### Multi-Segmental Spinal Plasticity Protocol

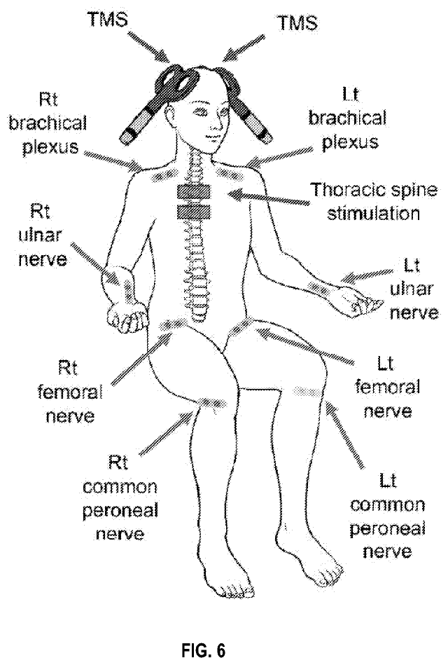

This validation study applied multisite paired corticospinal motoneuronal stimulation (MPCMS) using the system 100 (FIGS. 1-5) to elicit multi-segmental spinal plasticity. Specifically, 8 muscles which included right and left biceps brachii, first dorsal interosseous, quadriceps, and tibialis anterior were targeted in each individual. For each targeted muscle, corticospinal volleys evoked by either transcranial magnetic stimulation (TMS; for muscles in the upper extremities) or electrical stimulation on the thoracic spine (for muscles in the lower extremities) were timed to arrive at corticospinal-motoneuronal synapses of the targeted muscle before antidromic potentials elicited in motoneurons by electrical stimulation of a peripheral nerve. Eight individuals with chronic cervical SCI completed 40 sessions of MPCMS combined with exercise (FIGS. 6-8).

### Effects of MPCMS on Electrophysiological Recordings: Motor Evoked Potentials (MEPs)

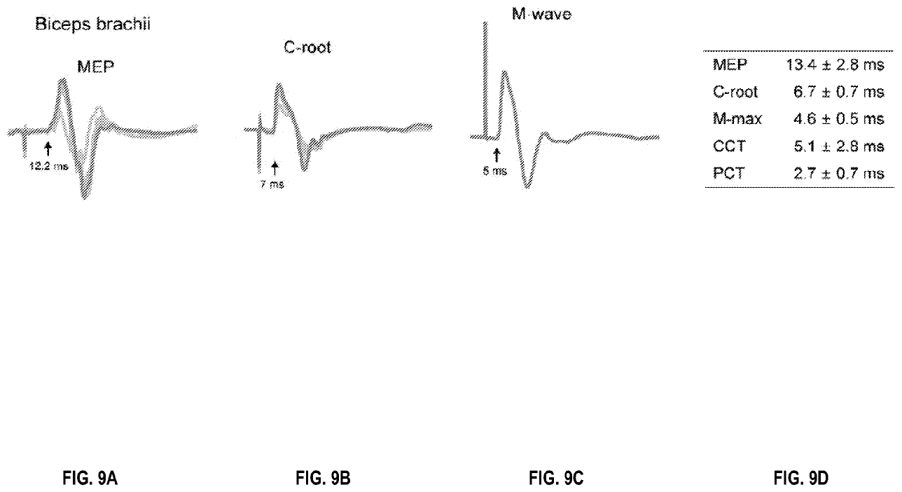

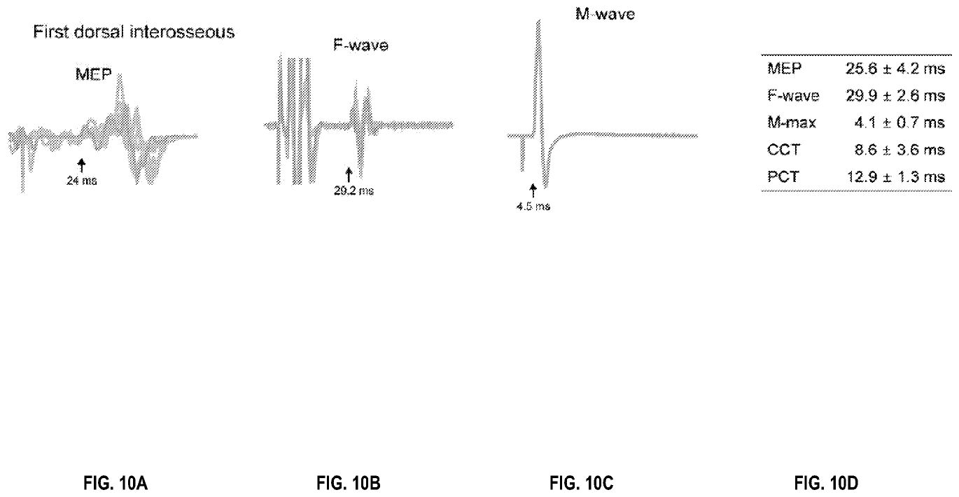

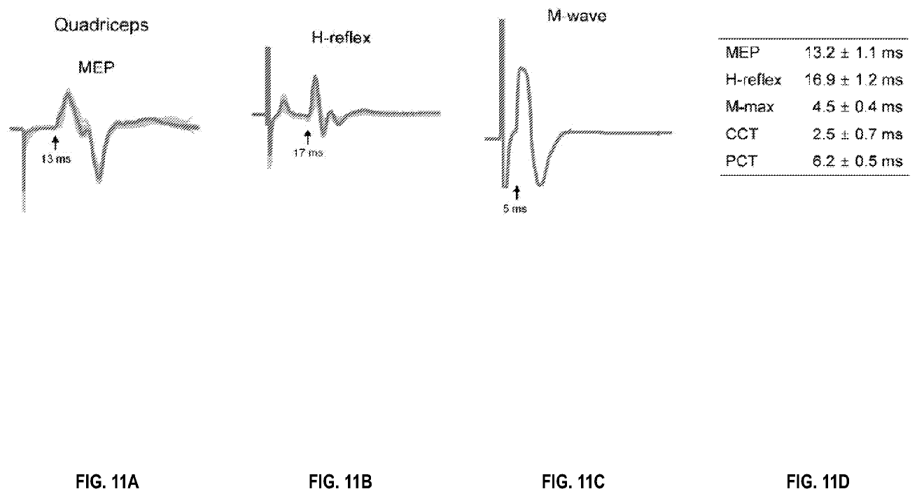

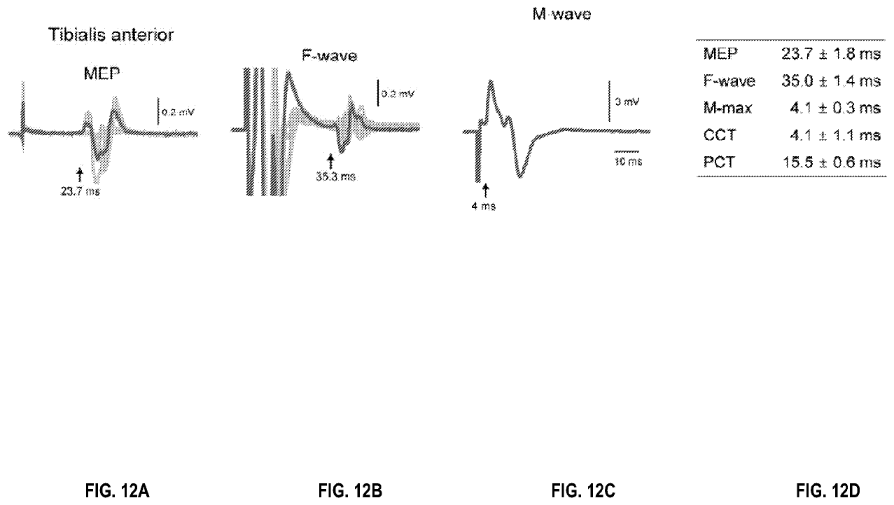

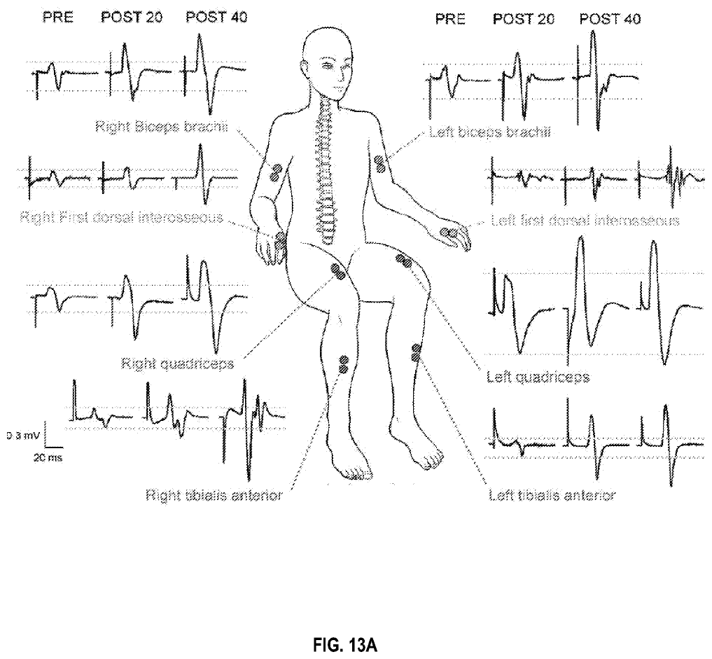

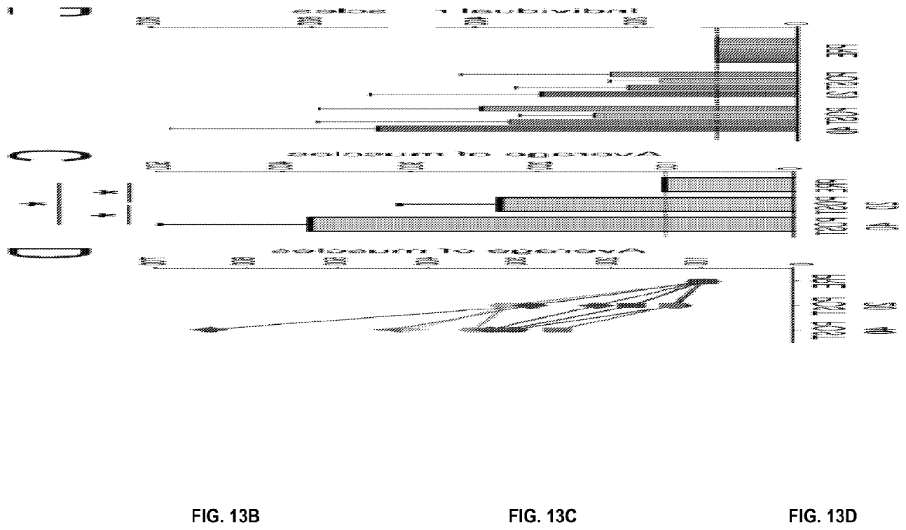

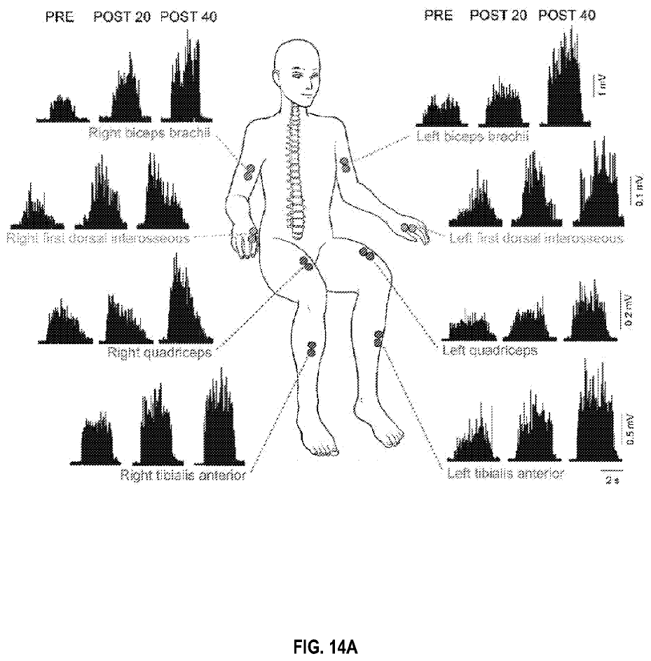

Referring to FIGS. 9A-13D, since MPCMS targets to strengthen corticospinal motoneuronal synapses, changes in transmission in the corticospinal pathway were first examined by assessing the size of MEPs before, after 20 sessions, and after 40 sessions of MPCMS combined with exercise. Stimulation used to evoke descending volleys was applied to assess MEPs: TMS for muscles in the upper extremities and electrical stimulation on the thoracic spine for muscles in the lower extremities. Participants with SCI showed increase in the size of MEPs after 20 sessions (p=0.006) and further increase after 40 sessions (p=0.007) in all targeted muscles. FIG. 9A shows raw MEP traces from representative participants in the biceps brachii (right: subject #6, left: subject #7), FIG. 10A shows the same for first dorsal interosseous (right: subject #6, left: subject #3), FIG. 11A shows the same for quadriceps (right: subject #2, left: subject #7), and FIG. 12A shows the same for tibialis anterior (right: subject #7, left: subject #1) muscles. Note that all participants showed increases in the amplitude of MEP after 20 sessions compared with baseline assessment and further increased after additional 20 sessions (FIG. 13D). There was no effect of muscles in the amplitude of MEP. Specifically, in biceps brachii, MEP size increased by 231.7±186.7% after 20 sessions and by 391.1±200.6% after 40 sessions. In first dorsal interosseous, MEP size increased by 168.3±64.4% after 20 sessions and by 252.9±89.0% after 40 sessions. In quadriceps, MEP size increased by 209.3±138.0% after 20 sessions and by 356.7±237.2% after 40 sessions. In tibialis anterior, MEP size increased by 316.1±210.9% after 20 sessions and by 517.0±259.1% after 40 sessions (FIG. 13B).

Additionally, MEP elicited by TMS in lower extremity (n=5) was assessed and similar results were observed. The amplitude of MEP increased after sessions of MPCMS combined with exercise (189.9±25.7%, p=0.004) and further increased after 40 sessions (328.3±62.0%, p=0.037). There was no effect of muscles in the amplitude of MEP. Specifically, in quadriceps, MEP size increased by 197.2±39.5% after 20 sessions and by 318.8±108.2% after 40 sessions. In tibialis anterior, MEP size increased by 182.7±19.9% after 20 sessions and by 337.8±94.1% after 40 sessions.

### Effects of MPCMS on Electrophysiological Recordings: Maximal Voluntary Contractions (MVCs)

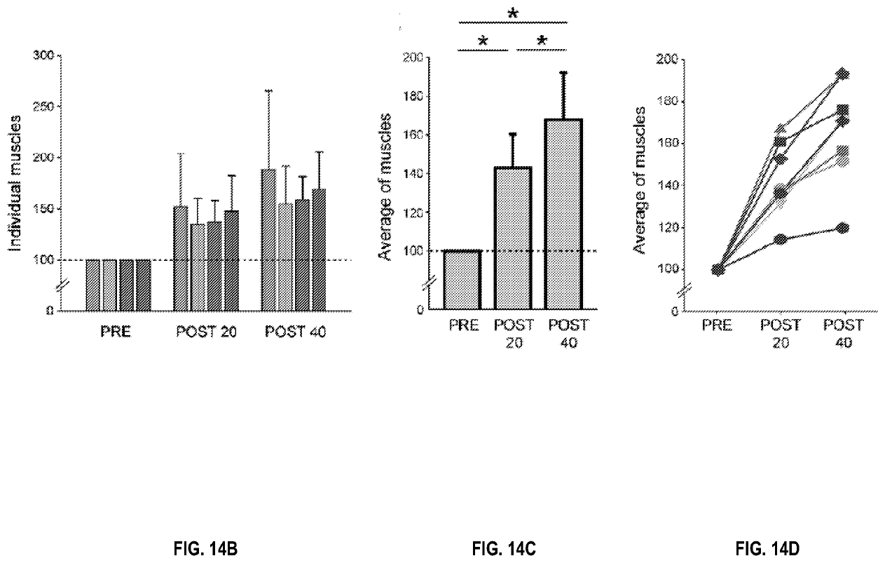

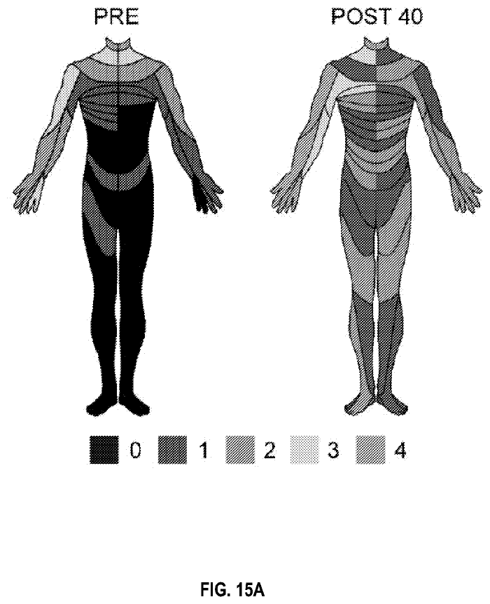

Referring to FIGS. 14A and 14B, the validation study next examined whether strengthening in transmission leads to changes in maximal voluntary contractions in the targeted muscles. FIG. 14A shows raw EMG traces during MVC from representative participants in the biceps brachii (right: subject #7, left: subject #8), first dorsal interosseous (right: subject #3, left: subject #7), quadriceps (right: subject #8, left: subject #6), and tibialis anterior (right: subject #2, left: subject #4) muscles. In all subjects, MVC increased in targeted muscles after 20 sessions of MPCMS combined with exercise and further increased after additional sessions. There was no effect of muscles in MVC. Specifically, in biceps brachii, MVC increased by 152.3±51.5% after 20 sessions and by 188.5±76.9%% after 40 sessions. In first dorsal interosseous, MVC increased by 135.2±25.0% after 20 sessions and by 154.8±37.0% after 40 sessions. In quadriceps, MVC increased by 137.1±20.6% after 20 sessions and by 158.4±23.0% after 40 sessions. In tibialis anterior, MVC increased by 147.3±35.1% after 20 sessions and by 169.4±36.2% after 40 sessions (FIG. 14B).

### Effects of MPCMS on Sensory and Motor Function

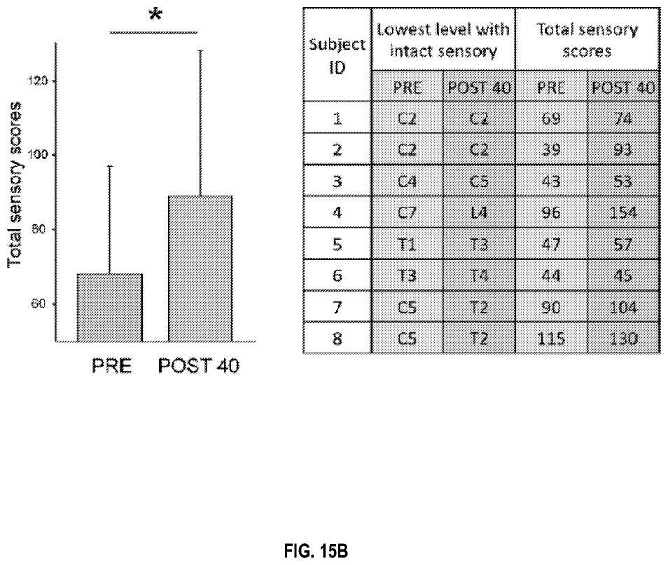

American Spinal Injuries Association Impairment Scale (AIS) was tested prior to the intervention and after 40 sessions of intervention. FIG. 15A shows examples of dermatomes for sensory scores before and after 40 sessions in a representative subject. Note that this subject fully restored in right hand and parts of upper limb (score of 4 shown in orange) and partially restored in left hand and upper limb. He did not have much sensation in his lower limbs but restored some sensation in most parts of lower limbs after intervention. All participants increased total sensory scores after intervention (p=0.015; FIG. 15B) and the lowest level with intact sensory (score of 4) changed to lower level in majority of participants (6 out of 8; FIG. 15B).

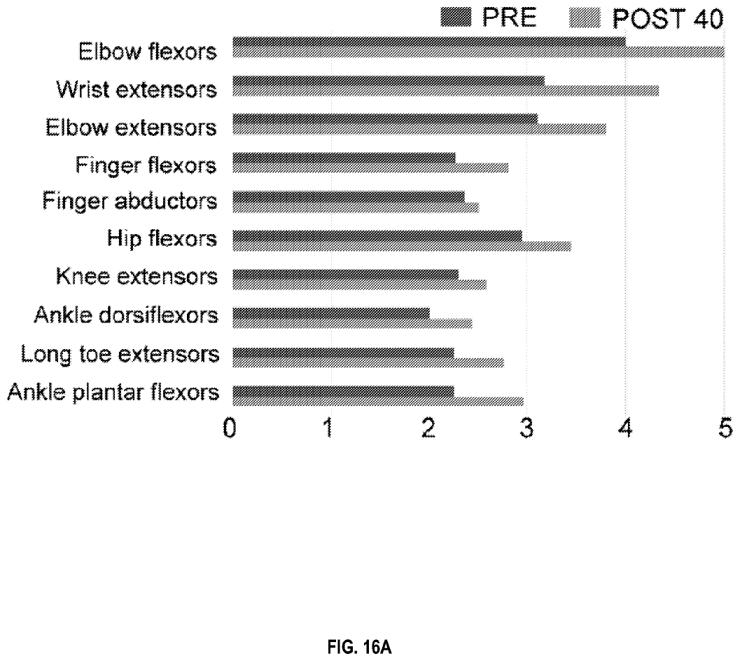

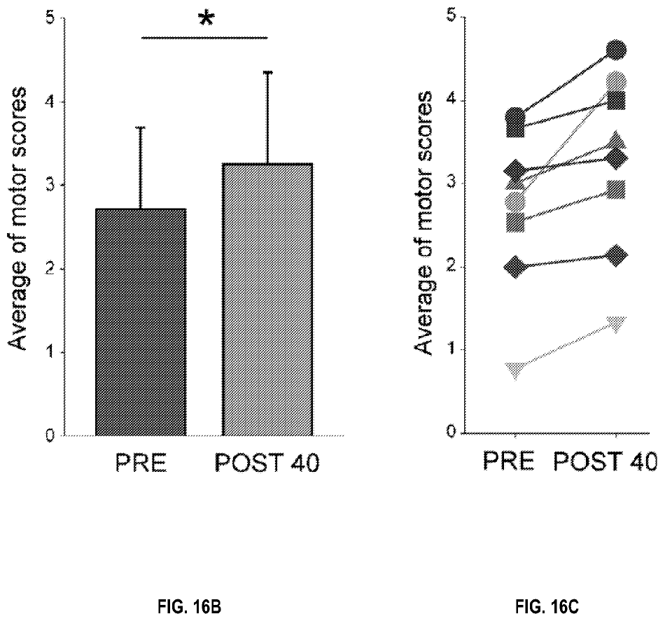

FIG. 16A shows motor scores of each muscle group before and after intervention. Note that motor score in all muscles increased after intervention as well as all participants increased mean motor scores in muscles with score of less than 5 at pre-assessment (p=0.013, FIG. 16B). Overall mean increased 0.5±0.4 points.

### Improvement in Grasping and Walking

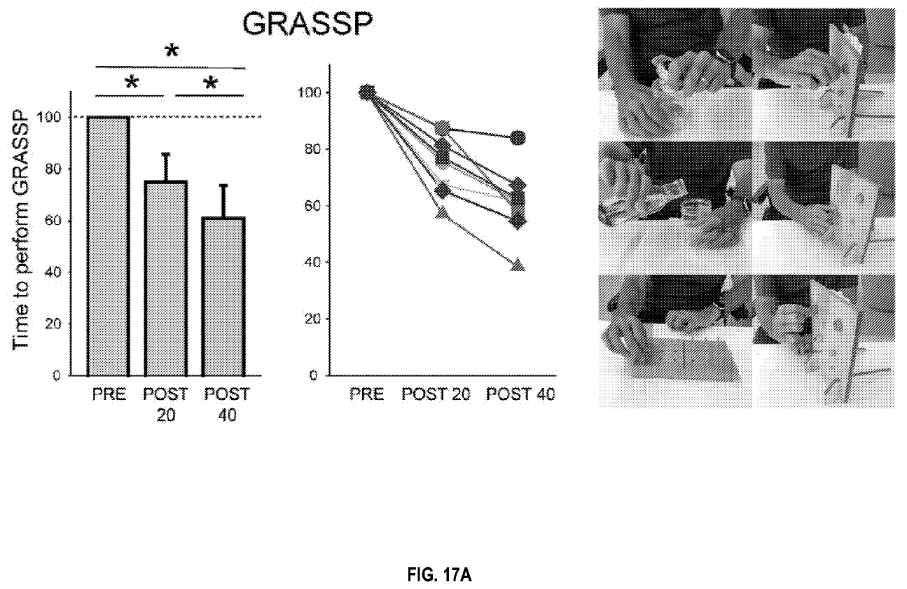

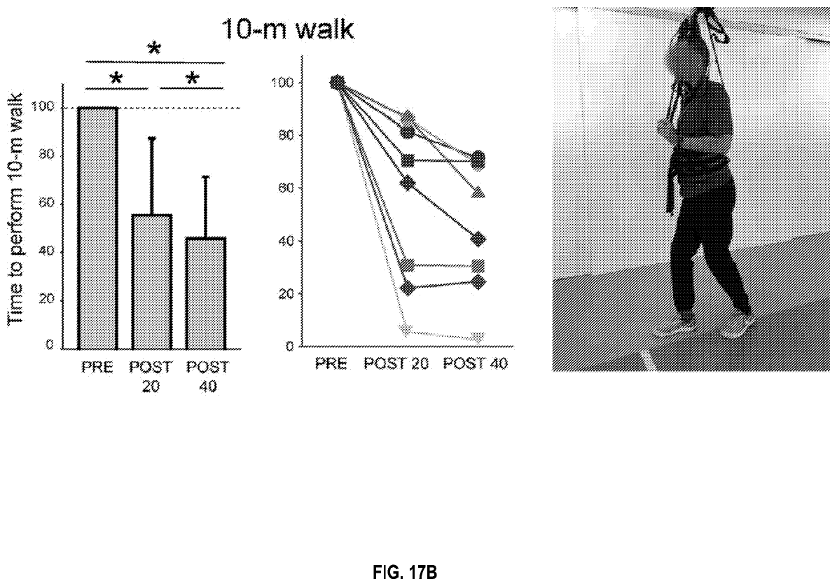

The validation study further examined whether changes in corticospinal transmission and muscle strength elicited by protocol affected functional performance of upper- and lower-limbs. For upper-limb function, tested gross (i.e. jar opening and water bottle tests) and fine (i.e. key, coin, nut and bolt, and nine-hole peg tests) grasping functions were tested using subcomponents of the Graded and Redefined Assessment of Strength, Sensibility and Prehension (GRASSP) test. The results showed that the time to perform GRASSP decreased after 20 sessions of MPCMS combined with exercise (25.2±10.8%, p=0.001) and further decreased after 40 sessions 39.0±12.7%, p=0.003). Note that all participants showed improved hand function after 20 sessions compared with baseline and further improved after additional 20 sessions (FIG. 17A). Similarly, 10-meter walk test used to test lower-limb function revealed that the time to perform 10-meter walk test decreased after 20 sessions of MPCMS combined with exercise (44.5±31.9%, p=0.017) and further decreased after 40 sessions 55.7±25.7%, p=0.04). Note that all participants showed improved walking speed during POST 20—compared with PRE-assessment and majority of participants (7 out of 8) further improved during POST 40-compared with POST 20-assessment (FIG. 17B). Notably, functional outcomes remained increased at the 6-months follow-up. GRASSP performance increased after 40 sessions of MPCMS+exercise (by 39.0±12.7%) and remained increased for 6 months (by 47.1±9.5%; p<0.001) compared with baseline. Similarly, 10-meter walk speed increased after 40 sessions of MPCMS+exercise (by 54.3±26.0%) and remained increased for 6 months (by 47.7±33.2%; p=0.009) compared with baseline.

### Improvement in Quality of Life

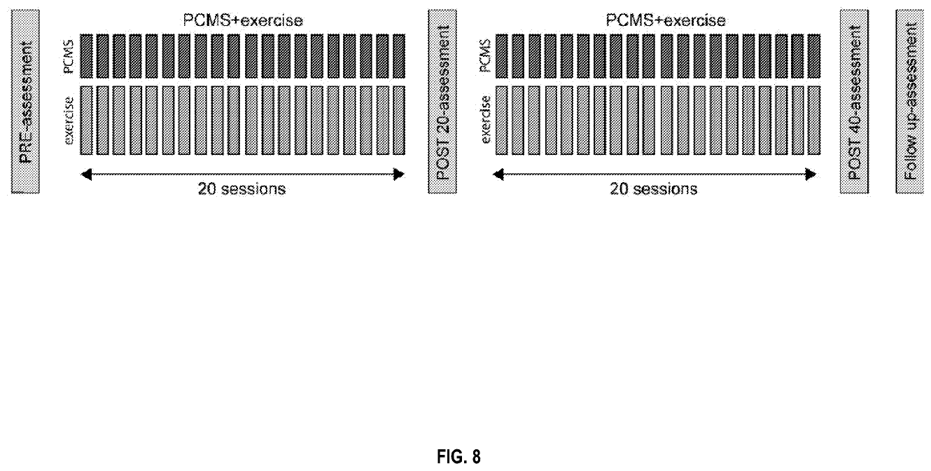

Finally, the validation study tested how these physiological and functional improvements were perceived by participants and affected their quality of life. 6 subdomains of the Spinal Cord Injury-Quality of Life (SCI-QOL) measurement were used including ambulation, basic mobility, fine motor functioning, and self-care for physical functioning as well as bowel management difficulties and bladder management difficulties for physical-medical health. Repeated-measures ANOVA showed an effect of FUNCTION (F_{1.2,8.6}=22.7, p=0.001) and TIME (F_{1,7}=6.8, p=0.03) but not in their interaction (F_{1.3,8.8}=0.7, p=0.4) on physical functioning subdomains. Post-hoc analysis revealed that self-reported function improved in ambulation (p=0.023) and self-care (p=0.036) sub-sections after the intervention while basic mobility (p=0.17) and fine motor (p=0.23) did not change significantly (FIG. 8). Repeated-measures ANOVA showed an effect of FUCTION (F_{1,7}=6.9, p=0.033) and TIME (F_{1,7}=13.6, p=0.008) but not in their interaction (F_{1,7}=3.9, p=0.09) on physical-medical health subdomains. Post-hoc analysis revealed that self-reported function improved in bladder difficulties (p=0.007) and bowel management (p=0.04) sub-sections after the intervention. Notably, self-reported functional changes for ambulation remained increased at the 6-months follow-up (p=0.04). However, changes in other sections returned close to baseline for self-care (p=0.4), bladder difficulties (p=0.3) and bowel management (p=0.3) sub-sections after 6 months.

### Materials and Methods

Participants. Eight individuals with chronic cervical SCI (mean age 45.9±16.4 years, 4 female) participated in the study. Written informed consent was obtained from all subjects for study participation for publishing their images or video in an online open-access publication. All procedures were approved by the local ethics committee at the Northwestern University in accordance with the guidelines established in the Declaration of Helsinki. Participants with SCI had a chronic (>1 year) injury between C1-C5. Two out of 8 individuals were categorized by the American Spinal Cord Injury Impairment Scale (AIS) as AIS C and the other 6 individuals were classified as incomplete AIS D.

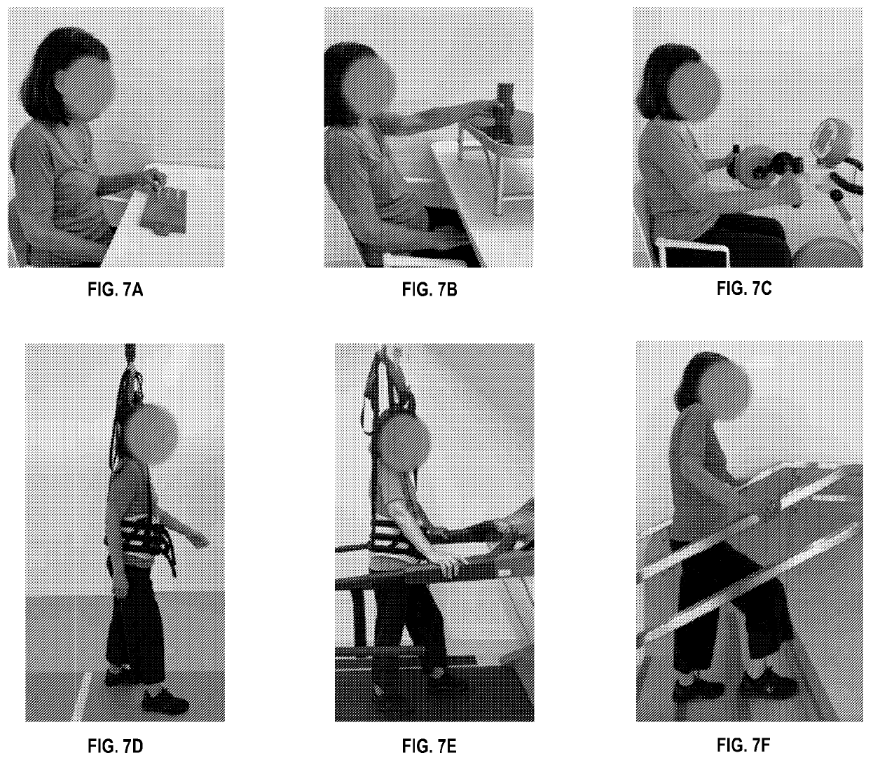

Study design. Individuals completed 40 sessions of MPCMS combined with exercise in 8-12 weeks (FIG. 6). Participants were asked to have 3-5 sessions per week. Studies have previously showed that the facilitatory effects of MPCMS on corticospinal excitability returned to baseline ˜60-80 min after the end of the stimulation. Thus, the exercise training (FIGS. 7A-F) lasted for 60 min and started immediately after MPCMS. In all subjects, the following measurements (FIGS. 8-12D) were tested prior to the intervention (PRE), after 20 sessions of intervention (POST 20) and after 40 sessions of intervention (POST 40: motor evoked potentials (MEP), maximal voluntary contractions (MVC), functional outcomes. American Spinal Injuries Association Impairment Scale (AIS) and self-administrated questionnaires, Spinal Cord Injury-Functional Index (SCI-FI), were tested prior to the intervention and after 40 sessions of intervention. All subjects returned for a 6-month follow-up session to examine the functional outcomes and SCI-FI.

Experimental set up. During testing of first dorsal interosseous, participants were seated in an armchair with both arms relaxed and flexed at the elbow by 90° with the forearm pronated and the wrist and forearm restrained by straps. When the biceps brachii was tested, individuals were seated in an armchair with a custom device attached to maintain the position of the tested arm with the shoulder and elbows flexed at 90°. When the quadriceps and tibialis anterior was tested, both feet were placed on a custom platform with the ankle flexed at 90° and restrained by straps.

Electromyography (EMG) recordings. EMG was recorded for 8 muscles which included right and left biceps brachii, first dorsal interosseous, quadriceps, and tibialis anterior through surface electrodes secured to the skin over the belly of each muscle (Ag-AgCl, 10 mm diameter). The signals were amplified, filtered (20-1000 Hz), and sampled at 10 kHz for offline analysis (CED 1401 with Signal software, Cambridge Electronic Design, Cambridge, UK).

TMS. Transcranial magnetic stimuli were delivered from the TMS device 120 of the system 100 (FIGS. 1-5) through either a figure-of-eight coil (used for muscles in the upper extremities; loop diameter, 7 cm; type number SP15560) or a double-cone coil (used for muscles in the lower extremities; type number 9902-00) with a monophasic current waveform. TMS was delivered to the optimal scalp position. The optimal scalp position for upper extremities was determined by moving the coil in small steps along the hand/arm representation of the primary motor cortex to find the region where the largest MEP could be evoked in both biceps brachii and first dorsal interosseous with the minimum intensity. The optimal scalp position for lower extremities was determined by moving the coil in small steps along the leg representation of the primary motor cortex to find the region where the largest MEP could be evoked in quadriceps and tibialis anterior with the minimum intensity. These scalp positions were saved using a stereotaxic neuro-navigation system (Brainsight 2, Rogue Research, Montreal, Canada) and used for assessments and MPCMS sessions. The TMS coil was held to the head of the subject with a custom coil holder, while the head was firmly secured to a headrest by straps to limit head movements.

Thoracic spine stimulation. Electrical stimulation of thoracic spine will be carried out by passing a high-voltage electrical current (200 μs) from the waveform generator 110 of the system 100 (FIGS. 1-5) between surface electrodes (7.5×13 cm) with the cathode between the spine of T3 and T4 and an anode 5-10 cm above it.

PNS. Supra-maximum electrical stimulation (200-1000 μs pulse duration) was delivered from the waveform generator 110 of the system 100 (FIGS. 1-5) to left and right brachial plexus at the Erb's point (to target left and right biceps brachii) and left and right ulnar nerve at the wrist (to target left and right first dorsal interosseous), left and right femoral nerve at inguinal crease (to target left and right quadriceps), and left and right common peroneal nerve under the head of the fibula (to target left and right tibialis anterior). The anode and cathode were 3 cm apart and 1 cm in diameter with the cathode positioned proximally. The stimuli were delivered at an intensity of 120% of the M-max for each muscle.

MPCMS. During MPCMS, 180 sets of stimuli were delivered every 10 s (˜30 min, 0.1 Hz) where two TMS coils were applied at the right and left arm/hand representation of primary motor cortex to generate descending volleys to all four targeting muscles in the upper extremities and each antidromic volley from four peripheral nerves was precisely timed to arrive at corticospinal-motoneuronal synapses of each muscle ˜1-2 ms after descending TMS volleys. Additionally, thoracic spine stimulation was applied to generate descending volleys to all four targeting muscles in the lower extremities and each antidromic volley from four peripheral nerves was precisely timed to arrive at corticospinal-motoneuronal synapses of each muscle ˜1-2 ms after descending thoracic spine stimulation volleys. TMS stimuli were delivered at an intensity of 100% of the maximum stimulator output during MPCMS. Thoracic spine stimulation was delivered at an intensity of 120% of the minimum intensity that can elicit thoracic MEPs≥50 μV in all four targeting muscles in legs. PNS stimuli were delivered at an intensity of 120% of the maximal motor response (M-max) for each muscle.

MPCMS interstimulus interval (ISO. The ISI between descending volleys (from TMS or thoracic spine stimulation) and antidromic PNS volleys was set to allow descending volleys to arrive at the presynaptic terminal of corticospinal neurons ˜1-2 ms before antidromic PNS volleys reached the motoneurons during MPCMS. The methods for timing the arrival of volleys at the spinal cord have been described previously. Briefly, the ISI was tailored to individual subjects based on conduction times calculated from latencies of MEPs, F-wave, and M-max (FIGS. 9A-12D). MEP latencies were recorded during isometric ˜10% of MVC of the target muscle to determine the shortest and clearest response for estimations. The onset latency was defined as the time when each response exceeded 2 SD of the mean rectified pre-stimulus activity (100 ms) in the averaged waveform. Peripheral conduction time (PCT) was calculated using the following equation:

PCT=(F-wave latency−M-max latency)×0.5

Central conduction time (CTT) was calculated using the following equation:

CCT=MEP latency−(PCT+M-max latency)

The latency of H-reflex was used instead when it is difficult to elicit F-waves. When it was not possible to record F-waves or H-reflex (i.e. biceps brachii)C-roots were stimulated with TMS at cervical spinous processes C5-6 as in a previous study. Then, CCT was calculated by adding to the latency from TMS of the C-root to 1.5 ms [estimated time of synaptic transmission plus conduction to the nerve root at the vertebral foramina] and subtracting from the MEP latency [MEP−(C-root+1.5)]. PCT was calculated by subtracting the M-max latency from the C-root latency and adding 0.5 ms, the estimated time of antidromic conduction time from the vertebral foramina to the dendrites [(C-root−M-max)+0.5)].

Exercise training. All participants exercised for −60 min immediately after MPCMS. Upper-limb exercises involved gross grasping, fine grasping, and hand cycle using an arm ergometer. During gross grasping, subjects were asked to reach and grasp a cylinder (6-cm diameter and 16-cm height, 100 gms), block (6.5×6.5×6.5 cm, 110 gms), cup (6-cm diameter at the bottom and 10-cm height, 50 gms) and lid (10-cm diameter and 1-cm height, 15 gms) randomly presented on a table located in front of them at a height of −20 cm. Then, subjects were asked to reach and grasp the object to put it back on the table. During fine grasping, participants performed similar movements but now they were asked to reach and grasp smaller objects (peg, bead, pinch pin, cube). These sets of movements were repeated 20 times for each object for 20 min with breaks as needed. During hand cycle, the arm ergometer was used for 10 minutes and grasping gloves were used as needed. Lower-limb exercises involved over-ground walking, treadmill walking, and stair climbing training. During walking, subjects used a harness connected to an overhead track and uses an active trolley system that automatically follows the patient as he or she walks. During treadmill walking, subjects walked at a speed of 0.1-0.3 m/s for 10 minutes using the ZeroG system. During stair climbing, subjects climbed up and down 4 steeps with 3 full repetitions. Note that although all participants were ambulatory a few of them were not able to walk without an assistive device (n=3).

MEPs. Cortically evoked motor potentials were measured in all 8 muscles with TMS. The maximal MEP size (MEP-max) was found in each subject for each muscle tested. The MEP-max was defined in all participants at rest by increasing stimulus intensities in 5% steps of maximal device output until the MEP amplitude did not show additional increase. For MEP measurements, TMS intensity was set at the intensity required to elicit an MEPs of 50% of MEP-max size on each muscle tested. Note that MEPs in one or both sides of quadriceps and/or tibialis anterior could not be elicited in some participants (3 out of 8) although they have voluntary activity in those muscles, likely due to higher thresholds. Therefore, subcortically evoked potentials were additionally measured with thoracic spine stimulation for leg muscles at the intensity defined for MPCMS and used for MEP comparisons in legs. All stimuli were delivered at 4s intervals (0.25 Hz). Twenty MEPs were recorded for each muscle and peak-to-peak MEP amplitude was measured in each trial and averaged. The same intensity was used during the pre, post, and follow-up assessments. In order to compare MEPs with similar background EMG activity between interventions, trials in which the background EMG activity (100 ms before the TMS stimulus artifact) was 2SD above the mean resting background EMG activity were excluded from the analysis; 4.7±4.1% of trials were excluded in SCI participants.

MVCs. Note that during MVC testing subjects were asked to perform three brief MVCs for 3-5 s with each of the muscles tested, separated by ˜30 s of rest. The order of tested muscles was randomized. MVCs were performed into index finger abduction for first dorsal interosseous, into elbow flexion for biceps brachii, into knee extension for quadriceps and into ankle dorsiflexion for tibialis anterior. The maximal mean EMG activity measure over a period of 1 s on the rectified response generated during each MVC was analyzed and the highest value of the three trials was used. Note that for these measurements, the mean background resting EMG activity obtained on each day (1 s before the MVC) was subtracted to facilitate comparisons of EMG amplitudes across different days.

Functional outcomes. For upper-limb function, gross (i.e. jar opening and water bottle tests) and fine (i.e. key, coin, nut and bolt, and nine-hole peg tests) grasping functions were tested using subcomponents of the Graded and Redefined Assessment of Strength, Sensibility and Prehension (GRASSP) test. During jar opening, subjects were asked to open a jar lid with a tested hand while holding the jar (7-cm diameter and 9-cm height) with the other hand as fast as possible. During the water bottle test, subjects were asked to lift a bottle (6-cm diameter and 20-cm height, filled with water ˜200 mL) from the table and pour water into the cup, approximately ¾ full. During the key test, subjects were asked to lift a key from the table, insert it in a lock, and turn it 90°. During coin test, subjects were asked to insert four coins to a coin slot one by one. During nut and bolt test, subjects were asked to screw four nuts onto bolts. During nine-hole peg test, subjects were asked to pick up nine pins and position each one of them into a reservoir. The instruction for all tests was to perform each task as fast and accurately as possible. The tasks were repeated 3 times for each hand. The distance and position between each subject's hand and the apparatus was recorded and maintained constant for pre- and post-assessments. For lower-limb function, we used the 10-meter walk test to assess walking speed. Overall, a stopwatch was used to measure the time to execute each task. Each task was repeated 3 times and the average was used. For 6-month follow-up, one participant did not try 10-m walk test because of leg pain but GRASSP was tested. All other participants were tested for both walking and GRASSP.

AIS. Motor and sensory function was evaluated with AIS by an experienced physical therapist specialized in SCI. For sensory scores, the lowest level with intact sensory scores (2 for light touch and 2 for pin prick) was identified and the scores below that level were summed to get total sensory scores. The level from pre-assessment was used for both pre- and post 40-assessments for comparison. For motor scores, the average of muscles that scored below 5 during the pre-assessment was calculated.

SCI-FI. Questionnaires on ambulation, basic mobility, fine motor, self-care, bladder difficulties and bowel management were used to assess physical functioning and quality of life. Raw total scores of each section was converted to T-scores. Note that higher scores indicate improvement in function for ambulation, basic mobility, fine motor and self-care whereas lower scores indicate better function of bowel and bladder.

Data analysis. Normal distribution was tested by the Shapiro-Wilk's test and homogeneity of variances by the Mauchly's test of sphericity. When sphericity could not be assumed, the Greenhouse-Geisser correction statistic was used. Repeated-measures analysis of variance was performed to determine the effect of TIME (pre-assessment, post 20-assessment, post 40-assessment) and MUSCLE (biceps brachii, first dorsal interosseous, quadriceps, tibialis anterior) on MEP size, background EMG activity before MEP stimulus artifact, and MVCs. The same repeated-measures analysis of variance was performed to determine the effect of TIME and LE-MUSCLE (quadriceps, tibialis anterior) on MEP size from TMS in muscles in the lower extremity. Data for right and left sides were averaged within each muscle for comparison. Repeated-measures analysis of variance was used to compare difference across TIME in functional outcomes. Right and left sides data for GRASSP were averaged within each subjects. Repeated-measures analysis of variance was used to determine the effect of TIME2 (pre-assessment, post 20-assessment) and FUNCTION on SCI-FI T-scores. FUNCTION includes ambulation, basic mobility, fine motor, self-care for motor categories and bowel and bladder difficulties and bowel management for bowel and bladder categories. Bonferroni post hoc tests were used to test significant comparisons. Paired t-tests were used to compare motor and sensory scores of AIS scores and SCI-FI results between pre- and post-40-assessments and between pre- and follow-up. Significance was set at p<0.05. Group data are presented as the means±SD in the text.

### Results

MEPs.

FIG. 13A shows raw MEP traces from representative participants in the biceps brachii (right: subject #6, left: subject #7), first dorsal interosseous (right: subject #6, left: subject #3), quadriceps (right: subject #2, left: subject #7), and tibialis anterior (right: subject #7, left: subject #1) muscles. Note that the amplitude of MEPs increased in targeted muscles after 20 sessions of MPCMS combined with exercise and further increased after additional 20 sessions.

Repeated-measures ANOVA showed an effect of TIME (F_{2,14}=33.6, p<0.001) but not MUSCLE (F_{3,21}=2.0, p=0.2) nor in their interaction (F_{6,42}=1.8, p=0.2) on MEP size. Post-hoc analysis revealed that the amplitude of MEP increased after 20 sessions of MPCMS combined with exercise (231.4±62.3%, p=0.006) and further increased after 40 sessions (379.4±108.9%, p=0.007; FIG. 13C). Note that all participants showed increases in the amplitude of MEP during POST 20—compared with PRE-assessment and further increases during POST 40-compared with POST 20-assessment (FIG. 13D). There was no effect of muscles in the amplitude of MEP. Specifically, in biceps brachii, MEP size increased by 231.7±186.7% after 20 sessions and by 391.1±200.6% after 40 sessions. In first dorsal interosseous, MEP size increased by 168.3±64.4% after 20 sessions and by 252.9±89.0% after 40 sessions. In quadriceps, MEP size increased by 209.3±138.0% after 20 sessions and by 356.7±237.2% after 40 sessions. In tibialis anterior, MEP size increased by 316.1±210.9% after 20 sessions and by 517.0±259.1% after 40 sessions (FIG. 13B).

MEP elicited by TMS in lower extremity (n=5) showed similar results. Repeated-measures ANOVA showed an effect of TIME (F_{2,8}=41.3, p=0.001) but not LE-MUSCLE (F_{1,4}=0.003, p=0.9) nor in their interaction (F_{1,4.1}=0.2, p=0.7) on MEP size. Post-hoc analysis revealed that the amplitude of MEP increased after 20 sessions of MPCMS combined with exercise (189.9±25.7%, p=0.004) and further increased after 40 sessions (328.3±62.0%, p=0.037). There was no effect of muscles in the amplitude of MEP. Specifically, in quadriceps, MEP size increased by 197.2±39.5% after 20 sessions and by 318.8±108.2% after 40 sessions. In tibialis anterior, MEP size increased by 182.7±19.9% after 20 sessions and by 337.8±94.1% after 40 sessions. No effect of TIME (F_{2,35}=2.4, p=0.2), MUSCLE (F_{1,35}=0.3, p=0.6) nor in their interaction (F_{2,35}=1.5, p=0.2) was found on mean background EMG activity measured prior to the TMS stimulus artifact.

### MVCs.

FIG. 13A shows raw EMG traces during MVC from representative participants in the biceps brachii (right: subject #7, left: subject #8), first dorsal interosseous (right: subject #3, left: subject #7), quadriceps (right: subject #8, left: subject #6), and tibialis anterior (right: subject #2, left: subject #4) muscles. Note that the MVC increased in targeted muscles after 20 sessions of MPCMS combined with exercise and further increased after additional 20 sessions.

Repeated-measures ANOVA showed an effect of TIME (F_{2,14}=52.7, p<0.001) but not MUSCLE (F_{3,21}=0.6, p=0.6) nor in their interaction (F_{1.8,12.7}=0.7, p=0.5) on MVC. Post-hoc analysis revealed that the amplitude of MEP increased after 20 sessions of MPCMS combined with exercise (143.0±17.3%, p=0.001) and further increased after 40 sessions (167.8±24.4%, p=0.003; FIG. 14C). Note that all participants showed increases MVC during POST 20—compared with PRE-assessment and further increases during POST 40-compared with POST 20-assessment (FIG. 14D). There was no effect of muscles in MVC. Specifically, in biceps brachii, MVC increased by 152.3±51.5% after 20 sessions and by 188.5±76.9%% after 40 sessions. In first dorsal interosseous, MVC increased by 135.2±25.0% after 20 sessions and by 154.8±37.0% after 40 sessions. In quadriceps, MVC increased by 137.1±20.6% after 20 sessions and by 158.4±23.0% after 40 sessions. In tibialis anterior, MVC increased by 147.3±35.1% after 20 sessions and by 169.4±36.2% after 40 sessions (FIG. 14B).

### Functional Outcomes

Repeated-measures ANOVA showed an effect of TIME (F_{2,14}=54.5, p<0.001) on GRASSP. Post-hoc analysis revealed that the time to perform GRASSP decreased after 20 sessions of MPCMS combined with exercise (25.2±10.8%, p=0.001) and further decreased after 40 sessions 39.0±12.7%, p=0.003). Note that all participants showed improved hand function during POST 20-compared with PRE-assessment and further improved during POST 40-compared with POST 20-assessment (FIG. 17A). Repeated-measures ANOVA showed an effect of TIME (F_{1.2,8.1}=22.2, p=0.001) on 10-meter walk test. Post-hoc analysis revealed that the time to perform 10-meter walk test decreased after 20 sessions of MPCMS combined with exercise (44.5±31.9%, p=0.017) and further decreased after sessions 55.7±25.7%, p=0.04). Note that all participants showed improved walking speed during POST 20—compared with PRE-assessment and majority of participants (7 out of 8) further improved during POST 40-compared with POST 20-assessment (FIG. 17B). Notably, functional outcomes remained increased at the 6-months follow-up. GRASSP performance increased after 40 sessions of MPCMS+exercise (by 39.0±12.7%) and remained increased for 6 months (by 47.1±9.5%; p<0.001) compared with baseline. Similarly, 10-meter walk speed increased after 40 sessions of MPCMS+exercise (by 54.3±26.0%) and remained increased for 6 months (by 47.7±33.2%; p=0.009) compared with baseline.

### AIS

FIGS. 15A-C show examples of dermatomes for sensory scores before and after 40 sessions in a representative subject. Note that this subject fully restored in right hand and parts of upper limb (score of 4 shown in orange) and partially restored in left hand and upper limb. He did not have much sensation in his lower limbs but restored some sensation in most parts of lower limbs after intervention. All participants increased total sensory scores after intervention (p=0.015; FIG. 15B) and the lowest level with intact sensory (score of 4) changed to lower level in majority of participants (6 out of 8; FIG. 15B).

FIGS. 16A-C show motor scores of each muscle group before and after intervention. Note that motor score in all muscles increased after intervention as well as all participants increased mean motor scores in muscles with score of less than 5 at pre-assessment (p=0.013; FIG. 16B). Overall mean increased points.

### SCI-FI

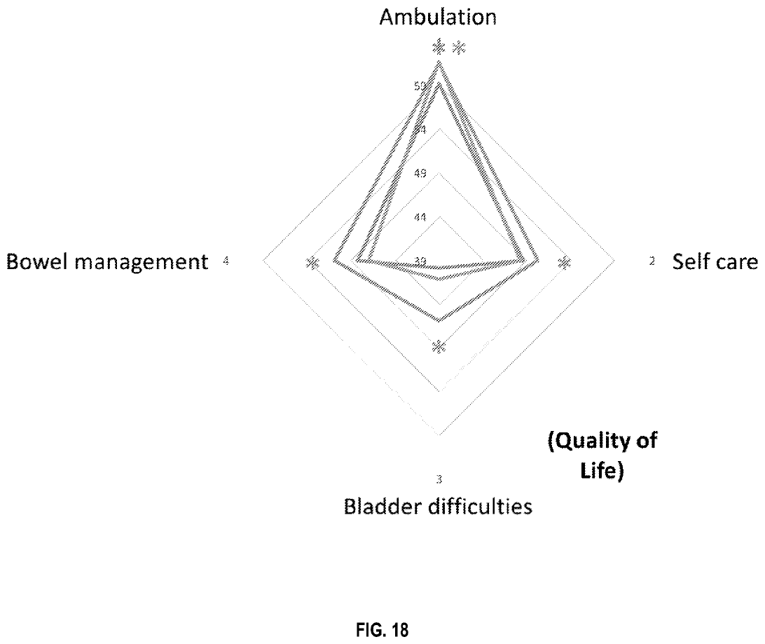

Repeated-measures ANOVA showed an effect of FUNCTION (F_{1.2,8.6}=22.7, p=0.001) and TIME (F_{1,7}=6.8, p=0.03) but not in their interaction (F_{1.3,8.8}=0.7, p=0.4) on motor categories. Post-hoc analysis revealed that self-reported function improved in ambulation (p=0.023) and self-care (p=0.036) sub-sections after the intervention while basic mobility (p=0.17) and fine motor (p=0.23) did not change significantly (FIG. 18). Repeated-measures ANOVA showed an effect of FUCTION (F_{1,7}=6.9, p=0.033) and TIME (F_{1,7}=13.6, p=0.008) but not in their interaction (F_{1,7}=3.9, p=0.09) on bowel and bladder categories. Post-hoc analysis revealed that self-reported function improved in bladder difficulties (p=0.007) and bowel management (p=0.04) subsections after the intervention. Notably, self-reported functional changes for ambulation remained increased at the 6-months follow-up (p=0.04). However, changes in other sections returned close to baseline for self-care (p=0.4), bladder difficulties (p=0.3) and bowel management (p=0.3) sub-sections after 6 months.

### Discussion

MPCMS was customized to target muscles in the upper and lower extremities simultaneously in individuals with incomplete cervical SCI. It was found that clinical functional outcomes improved in both hand function and walking by 48% after 40 sessions. Notably, this is reflected in self-reported improvements in quality of life in all eight participants. It was found that both motor and sensory scores of ASIA increased after protocol. Above improvements in clinical outcomes were accompanied by physiological changes such as ˜279% increase in the amplitude of motor evoked potentials of all muscles targeted by MPCMS. Maximal voluntary contractions also increased ˜68% in all muscles targeted by MPCMS. The functional improvement as well as improvements in quality of life persisted for 6 months, indicating that MPCMS induces stable plastic changes in the spinal synapses. These findings demonstrate that targeted non-invasive stimulation of multiple spinal synapses might represent an effective strategy to facilitate exercise-mediated recovery that can lead to improved function and quality of life in humans with spinal cord injury.

Recent studies have tested the effect of paired neural stimulation paradigms on exercise-induced recovery from SCI. Here, it was found that 40 sessions of MPCMS combined with upper and lower limb exercise improve fine and gross hand function and walking ability in individuals with chronic incomplete SCI. Compared to previous MPCMS protocol targeting one muscle for 10 sessions, current protocol were able to increase the same outcome measurements to a greater extent. Specifically, this validation study showed increase in MEP by 65% in a targeted muscle after 10 sessions in another study and it further increased by 131% after 20 sessions and 279% after 40 sessions in 8 targeted muscles on average. Increase in MVC was by 48% in a targeted muscle after 10 sessions whereas in the current protocol the increase was by 43% after 20 sessions and 68% after 40 sessions in 8 targeted muscles on average. The increase was not as pronounced in MVC and it was speculated that it might be because in the previous study, the weaker side was targeted with residual function present in each subject while in the current study, the same 8 muscles were targeted in all participants. Therefore, some participants in the current protocol had stronger strength in some muscles at baseline measurements compared to previous study participants (MVCs for biceps brachii: previous=0.47, current=0.43; FDI: previous=0.05, current=0.16; tibialis anterior: previous=0.05, current=0.1 mV), which may explain less pronounced increase in MVC after the protocol because measurements in each subjects were compared with their own baseline. Indeed, a negative correlation was found between baseline MVC and increase in MVC after 40 sessions (r=−0.78, p=0.03). Considering profound impact of SCI on quality of life, it is particularly encouraging that all study participants rated their quality of life with higher total scores after the MPCMS protocol applied using the present system 100. Specifically, subsections of ambulation, self-care, bladder difficulties, and bowel management showed increase after 40 sessions. Although changes in quality of life were not measured in anotherstudy of 10 sessions, a recent study on paired associative stimulation showed that the protocol enhanced strength and walking speed without detecting changes in self-reported function in self-care or mobility after 28 sessions (˜8 weeks) when applied on legs in individuals with chronic incomplete cervical SCI. Interestingly, the same stimulation protocol was applied on hands for an individual with cervical SCI for longer term (˜47 weeks) and the subject reported improved function for mobility as well as self-care. Although it is difficult to compare the magnitude of the effect across different stimulation protocols because the types of training, methods to quantify improvements, and characteristics of participants might differ, this disclosure would like to emphasize that participants with incomplete cervical SCI reported improvement in quality of life only after 40 sessions (8-12 weeks). While improvements in ambulation category persisted in 6-month follow-up questionnaires as well as functional measurements during GRASSP and walking, scores of other sub-categories of questionnaires returned back to baseline at 6-month measurements. Improvements in bowel and bladder function were also reported in studies using other types of neuromodulation such as epidural stimulation and transcutaneous stimulation with its effect lasting at least for up to 3 months. These results suggest that the effects of bowel and bladder function were less persistent compared to its effect on motor function after the protocol. In fact, self-care section of questionnaires involved numerous questions related to bowel and bladder function, which explains decreased scores for self-care at follow-up. Note that recent review on the efficacy of activity-based therapy interventions on quality of life reported no positive effects on quality of life outcomes in people with SCI.

It is speculated that MPCMS strengthens the connections between corticospinal neurons and motoneurons and increases motor output by enhancing synaptic plasticity, which persists after the protocol. This is supported by results on functional outcomes improved by ˜47% after 40 sessions in gross and fine hand motor tasks and walking speed (compare to ˜20% improvement after 10 sessions) and lasted for at least up to ˜6 months. Note that functional improvements were further enhanced without plateauing, which support the longer application of the protocol to explore its potential effect in individuals with SCI in future studies.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

1. A system, comprising:
a plurality of post-synaptic electrodes in communication with a waveform generator;
a processor in communication with a memory and the waveform generator, the memory including instructions, which, when executed, cause the processor to:
periodically apply a pre-synaptic stimulus having a pre-synaptic time of arrival to a motor cortex pathway of a body; and
periodically apply, by the waveform generator, a plurality of post-synaptic stimuli each having a respective post-synaptic time of arrival to a respective peripheral nerve of a plurality of peripheral nerves of the body such that an interstimulus interval between the pre-synaptic time of arrival of the pre-synaptic stimulus at a synapse of a corticospinal-motoneuronal neuronal pair of the body and each post-synaptic time of arrival of the each post-synaptic stimulus at the synapse is within a predetermined range.

2. The system of claim 1, wherein each post-synaptic electrode of the plurality of post-synaptic electrodes is configured for electrical communication with a respective peripheral nerve of the plurality of peripheral nerves of the body and the waveform generator.

3. The system of claim 1, wherein the predetermined range of the interstimulus interval is preferably between 1 millisecond and 2 milliseconds.

4. The system of claim 1, further comprising:
a transcranial magnetic stimulation (TMS) device in association with the processor, wherein the TMS device is operable to apply the pre-synaptic stimulus to the motor cortex pathway.

5. The system of claim 4, wherein the memory includes instructions, which, when executed, further cause the processor to:
provide a control input to the TMS device that causes the resultant pre-synaptic stimulus to arrive at the synapse at the pre-synaptic time of arrival.

6. The system of claim 1, wherein the system generates the pre-synaptic stimulus within the spinal cord of the body by stimulating the corticospinal pathway by application of a magnetic field parallel to a skull of the body, wherein the applied magnetic field is configured to induce an action potential within the corticospinal neuron that terminates in a pre-synaptic cell of the corticospinal-motoneuronal neuronal pair of the body.

7. The system of claim 1, further comprising:
a pre-synaptic electrode in association with the waveform generator, wherein the pre-synaptic electrode is configured to apply the pre-synaptic stimulus to a descending motor pathway.

8. The system of claim 7, wherein the memory includes instructions, which, when executed, further cause the processor to:
provide a control input to the waveform generator that causes the resultant pre-synaptic stimulus to arrive at the synapse at the pre-synaptic time of arrival.

9. The system of claim 1, wherein the system generates the pre-synaptic stimulus within a corticospinal neuron of the motor cortex pathway of the body by application of the pre-synaptic stimulus to a thoracic spine of the body to stimulate a descending motor pathway that terminates in a pre-synaptic cell of the corticospinal-motoneuronal neuronal pair of the body.

10. (canceled)

11. The system of claim 1, wherein the memory includes instructions, which, when executed, further cause the processor to:
provide a plurality of control inputs to the waveform generator that cause each resultant post-synaptic stimulus of the plurality of post-synaptic stimuli to arrive at the synapse at their respective post-synaptic times of arrival.

12. The system of claim 11, wherein the memory further includes instructions, which, when executed, further cause the processor to:
provide a control input to the waveform generator that adjusts a post-synaptic pulse initiation time of the post-synaptic stimulus of the plurality of post-synaptic stimuli based on a length of an associated peripheral nerve of the plurality of peripheral nerves such that the interstimulus interval is within the predetermined range.

13. The system of claim 1, wherein the system generates a post-synaptic stimulus of the plurality of post-synaptic stimuli within a respective peripheral nerve of the plurality of peripheral nerves of the body by application of an electrical waveform to a peripheral limb of the body to stimulate the peripheral nerve that terminates in a post-synaptic cell of the corticospinal-motoneuronal pair of the body.

14. The system of claim 1, wherein the system applies the post-synaptic stimuli such that the pre-synaptic stimulus from the motor cortex pathway arrives at the synapse 1-2 ms before each post-synaptic stimulus of the plurality of post-synaptic stimuli from the plurality of peripheral nerves.

15. (canceled)

16. The system of claim 1, wherein the memory includes instructions, which, when executed, further cause the processor to:
measure a plurality of latency values associated with a plurality of targeted peripheral nerves and the motor pathway;
determine a peripheral conduction time (PCT) and a central conduction time (CCT) based on the plurality of latency values; and
select a post-synaptic pulse initiation time of the post-synaptic stimulus such that the interstimulus interval between the pre-synaptic time of arrival and the post-synaptic time of arrival at a synapse is within the predetermined range interval.

17. A method of treating a subject, the method comprising:
(a) identifying two or more peripheral nerves innervating at least two different muscle sites in the subject and forming two or more peripheral nerve-muscle pairings;
(b) identifying two or more corticospinal-motoneuronal connections each comprising a corticospinal neuron connected at a synapse with each peripheral nerve in each of the peripheral nerve-muscle pairings;
(c) calculating a peripheral conduction time (PCT) and a central conduction time (CCT) for the each of the peripheral nerve-muscle pairings;
(d) periodically applying a first stimulus to a location in the central nervous system (CNS) in the subject such that the first stimulus triggers a descending signal in at least one corticospinal neuron in the corticospinal-motoneuron connections; and
(e) periodically applying a second stimulus to each of the two or more peripheral nerves such that the second stimulus triggers an ascending signal in the each of the two or more peripheral nerves,
wherein, each ascending signal and each descending signal arrive at the synapse of each corticospinal-motoneuronal connections and the descending signal arrives at a pre-determined interstimulus interval (ISI) prior to the arrival of the ascending signal.

18-19. (canceled)

20. The method of claim 17, wherein the peripheral conduction time (PCT) for each peripheral nerve-muscle pairing is calculated using the following equation: PCT=(F-wave latency−M-max latency)×0.5.

21. The method of claim 17, wherein the central conduction time (CCT) for each peripheral nerve-muscle pairing is calculated using the following equation: CCT=MEP latency−(PCT+M-max latency).

22-24. (canceled)

25. The method of claim 17, wherein the interstimulus interval (ISI) is about 0-5 milliseconds.

26. The method of claim 25, wherein the ISI is about 1-2 milliseconds.

27. The method of claim 17, wherein paired sets of first and second stimuli are applied at a frequency of about 0.1 Hz for about 30 minutes.
